(19) United States
(12) Patent Application Publication
(10) Pub. No.: US 2016/0331662 A1
(43) Pub. Date: Nov. 17, 2016

(54) Compositions that Inhibit and Prevent the Formation of Dental Caries and Methods of Using the Same

(71) Applicant: UAB RESEARCH FOUNDATION, Birmingham, AL (US)

(72) Inventors: Champion Deivanayagam, Birmingham, AL (US); Sangeetha Purushotham, San Diego, CA (US)

(73) Assignee: UAB Research Foundation, Birmingham, AL (US)

(21) Appl. No.: 15/109,790

(22) PCT Filed: Jan. 9, 2015
(86) PCT No.: PCT/US2015/010794

§ 371 Date: Jul. 5, 2016

Related U.S. Application Data
(60) Provisional application No. 61/925,474, filed on Jan. 9, 2014.

(51) Int. Cl. A61K 8/64; A61K 8/73; A61Q 11/02; A61Q 11/00

(52) U.S. Cl. CPC A61K 8/64; A61Q 11/00; A61K 8/73; A61Q 11/02; A61K 2800/92

(57) ABSTRACT

The present invention is related to the inhibition of binding of oral streptococci to the tooth surface. Compositions and methods for preventing, inhibiting and/or treating the formation of dental caries, and methods of identifying compounds that prevent, inhibit and/or treat the formation of dental caries are provided.

### RELATED APPLICATION INFORMATION

This application claims priority under 35 U.S.C. §119(e) to U.S. Provisional Patent Application No. 61/925,474, filed Jan. 9, 2014, the disclosure of which is incorporated herein by reference in its entirety.

### STATEMENT OF GOVERNMENT SUPPORT

This invention was made with government support in part under Grant No. DE017737 awarded by the National Institutes of Health and the National Institute of Dental and Craniofacial Research. The government has certain rights in the invention.

### STATEMENT REGARDING ELECTRONIC FILING OF A SEQUENCE LISTING

A Sequence Listing in ASCII text format, submitted under 37 C.F.R. §1.821, entitled 5656-58WO_ST25.txt, 2,496 bytes in size, generated on Jan. 9, 2015 and filed via EFS-Web, is provided in lieu of a paper copy. The Sequence Listing is incorporated herein by reference into the specification for its disclosures.

### FIELD OF THE INVENTION

The present invention relates to compositions that interfere with the adherence of oral streptococci to the tooth surface, and consequently can prevent the colonization and infection that lead to tooth decay. Galβ1-3-GalNac has been identified to be an inhibitor of adherence of oral streptococci to the tooth surface. The compositions of the present invention, methods of using the same and methods of identifying compositions that interfere with the adherence of oral streptococci to the tooth surface are related to the prevention and inhibition of the formation of dental caries.

### BACKGROUND OF THE INVENTION

The attachment of bacteria to human tissue and other surfaces within the oral cavity is thought to be an essential first step in pathogenesis, and microbes utilize surface proteins (pili, fimbrae) to effectively adhere to a variety of molecules and surfaces. While the oral cavity is home to a number of microbes this study focuses on oral streptococci, where the mutans streptococci (S. mutans, S. sobrinus), are the known etiological agents in dental caries, whereas the viridians streptococci (S. gordonii, S. sanguis) are considered to be commensal flora. Among the surface proteins on oral streptococci, Antigen I/II (AgI/II) homologs (also known as P1, PAc, SpaP, SR in S. mutans, SspA and SspB in S. gordonii, Pas in S. intermedius, etc. are the most extensively studied. These AgI/II homologs adhere to tooth immobilized salivary agglutinin (SAG) secreted by salivary glands. Typically, AgI/II homologs carry a signal sequence at the N-terminus, followed by the alanine-rich (A), variable (V) and proline-rich (P) regions, succeeded by the C-terminal region and the membrane spanning domain that anchors to the bacterial cell wall (FIG. 1A). In earlier studies, two SAG adherence regions have been identified on AgI/II: one, the V-region, at the apex of the molecule (A₃VP); and the other, the C₁C₂ region, at the C-terminus (C₁₂₃), specifically the C₁ and C₂ domains that adopt the DEv-IgG fold, a variant of the classical IgG-fold, near to where AgI/II is attached to the streptococcal cell surface (FIG. 1B). More importantly we determined that these two regions adhere to SAG in a non-competitive manner, indicating the presence of two different surfaces on SAG, pointing towards bacterial heterogeneity (multivalency) in adherence. Thus far all these interactions have been studied with purified SAG (some groups have now begun to address SAG as Gp340) extracted from single or multiple donors, and in some cases with saliva itself.

SAG is a large glycoprotein complex that contains glycoprotein 340 (Gp340), sIgA and an unknown 80 kDa protein. Among these the major component Gp340 is known to aggregate several species of bacteria, including mutans, viridan streptococci and H. pylori and is thereby considered an innate immune response factor. Gp340 orthologs are observed in various mammalian species including mouse, rabbit, rat, pig, cow and rhesus monkey. Gp340 is a 340 kDa protein that contains 14 SRCR (scavenger receptor cysteine rich) domains, 2 CUB (C1r/C1s Uegf Bmp1) and one ZP (zona pellucida) domain (FIG. 2). The 13 SRCR domains are present in tandem at the N-terminus, followed by an intriguingly nested 14^{th} SRCR domain between two CUB domains, with a ZP domain at the C-terminus. The SRCR domains are interspersed with regions termed SID, an acronym for the SRCR interspersed domains. Except between the 4^{th} and the 5^{th} SRCR domain, all other tandem repeats contain the SID domain. These SRCR domains belong to an ancient class of proteins and are present in protozoan parasites like Cryptosporidium, Toxoplasma, Plasmodium and in the algae Chlamydomonas. They also appear in the entire animal kingdom beginning with sponges, and are highly conserved, where a single SRCR domain usually contains 100-110 amino acids. The SRCR domains of Gp340 were recently shown to aid in transcytosis of HIV into vaginal epithelial cells. This highlights the role of the Gp340 SRCR domains in infection, where it serves as a portal of entry into the host for both bacteria and viruses that result in various human diseases. Therefore, Gp340 and its major constituent the SRCR domain has now become the focus of a number of recent reviews that highlight the importance of Gp340 in bacterial and viral pathogenesis.

In a systematic study conducted with various oral streptococci, Loimaranata et al. classified the bacterial recognition properties of Gp340 into three different groups, where group I strains both aggregated by and adhered to gp340, group II preferentially adhered, and group III preferentially aggregated. Using a peptide based approach, Bikker et al. identified a consensus peptide SRCRP2 (QGRVEVLYRGSWGTVC, SEQ ID NO: 1) derived from the 14 SRCR domains of Gp340, which aggregated several species of bacteria, and also inhibited the adherence of AgI/II to SAG. In a subsequent study using alanine scanning, the most important residues involved in aggregation were deduced to reside within the ‘VEVLXXXXW’ (SEQ ID NO:2) motif. In these studies, the SID domains that are predicted to host the glycosylation sites were classified into two different groups namely, SID20 and SID22 based on sequence homology, and neither one displayed aggregation nor adherence to bacteria. However, it is not believed to date that the interaction between the oral streptococci and the SRCR domains has been characterized.

The ability to adhere strongly to human receptors within the oral cavity is a necessity for bacterial survival, or else they will be washed into the acidic gut. Bacteria that colonize the oral cavity have multiple proteins on its surface for specific adherence to human receptors. The present invention is related to the S. mutans surface receptor AgI/II and its homologs, for example, S. gordonii SspB, and the development of inhibitors of the interaction of AgI/II and its homologs with Gp340, which is considered to be the first step in adherence of oral streptococci to the tooth surface. The adherence of oral streptococci subsequently leads to colonization and infection, and among these the mutans streptococci are known etiological agents in dental caries. Thus, compositions that can inhibit the adherence of oral streptococci to the tooth surface can provide an avenue for the development of compositions suitable for preventing the development of dental carries.

### SUMMARY OF THE INVENTION

The present invention utilizes the inhibition of the interaction between AgI/II, and/or its homologs, and SAG, more particularly with the SRCR domains found on Gp340 within the SAG complex which provides the basis for the compositions and methods of the present invention.

Thus, in an aspect of the invention, provided is a composition for the prevention and/or inhibition of the formation of dental caries in a subject. In another aspect of the invention, provided is a composition for the prevention and/or inhibition of the formation of denture plaques in a subject.

In yet another aspect of the invention, the compositions of the present invention comprise inhibitors of the interaction of AgI/II, and/or its homologs, with SAG. In a particular aspect of the invention, the inhibitor of the composition is a Galβ1-3-GalNac glycan. In another particular aspect of the invention, the inhibitor is a peptide. In yet another aspect of the invention, inhibitor binds to AgI/II and/or its homologs.

In yet another aspect of the invention, provided are formulations that comprise the composition for the prevention and/or inhibition of the formation of dental caries in a subject. In a further aspect of the invention, provided are formulations that comprise the composition for the prevention and/or inhibition of the formation of denture plaques in a subject.

In yet another aspect of the invention, provided is a method for inhibiting the interaction of AgI/II, and/or its homologs, with SAG, the method comprising the administration of the composition, compositions or formulations of the present invention.

In yet another aspect of the invention, provided is a method for preventing, inhibiting and/or treating the formation of dental caries in a subject, the method comprising the administration of the composition, compositions or formulations of the present invention.

In yet another aspect of the invention, provided is a method of identifying compounds that inhibit the interaction of AgI/II, and/or its homologs, with SAG.

In yet another aspect of the invention, provided is a method of identifying compounds for preventing, inhibiting and/or treating the formation of dental caries in a subject.

The foregoing and other objects and aspects of the present invention are explained in detail in the drawings and specification set forth below.

### BRIEF DESCRIPTION OF THE DRAWINGS

FIG. 1A depicts the primary sequence layout of S. mutans UA159 AgI/II and S. gordonii DL1 SspB including the extents of the recombinant fragments used herein.

FIG. 1B depicts the structure for AgI/II as derived from crystal structures of A₃VP₁ and C₁₂₃, and from electron microscopy.

FIG. 2 shows a schematic representation of the primary sequence layout of Gp340 from human saliva depicting the overall architecture.

FIG. 3 depicts recombinantly expressed and purified fragments of S. gordonii DL1 FL^{SspB}, A₃VP₁^{SspB}, and C₁₂₃^{SspB} on a 12.5% SDS-PAGE gel stained with coomassie blue.

FIG. 4 shows confocal microscopic images displayed the interaction between S. mutans UA159 (stained with blue DAPI) and iSRCRs (stained with green Anti-His tag Alexa fluor 488 antibody). The observed green fluorescence indicated the adherence of both iSRCR₁ (panel A) and iSRCR₁₂₃ (panel B) with S. mutans, where iSRCR₁₂₃ adhered more profoundly. S. mutans displayed counterstaining only with DAPI in the absence of iSRCRs (panel C).

FIG. 5 shows confocal microscopic images of S. gordonii DL1 interaction with iSRCRs similar to FIG. 4, panel A. Even in these images, iSRCR₁₂₃ displayed more profound interaction compared to iSRCR₁.

FIG. 6 depicts histograms constructed from FACS analysis describe the interaction of iSRCR₁ and iSRCR₁₂₃ with S. mutans in Panel A and with S. gordonii in Panel B.

FIG. 7 shows aggregation of (panel A) S. mutans UA159 and (panel B) S. gordonii DL1 cells in the presence of iSRCRs or SAG. Bacterial cells in buffer alone were used as control. The results are plotted as percentage of aggregation measured at OD₇₀₀ at 5 minute intervals for 1 hour. Difference in aggregation detected between groups were analyzed using One-way ANOVA, where *P<0.05 was considered significant, and error bars represent the standard deviation.

FIG. 8 depicts aggregation of S. mutans UA159 and S. gordonii DL1 cells in the presence of SRCRP2 peptide at different concentration. Bacterial cells in buffer alone were used as control. The results are plotted as percentage of aggregation measured at OD₇₀₀ at 5 minute intervals for 1 hour. Difference in aggregation detected between groups were analyzed using One-way ANOVA, where *P<0.05 was considered significant, and error bars represent the standard deviation.

FIG. 9 shows binding of SRCRs with different concentrations (1 ng-1 μg) of (A) AgI/II of S. mutans and (B) SspB of S. gordonii analyzed using ELISA. The dotted line (---) represent iSRCR₁ and the bold line (-) represent iSRCR₁₂₃. The data shows FL^{AgI/II} and FL^{SspB} binds to iSRCR₁ and iSRCR₁₂₃ with higher affinity compared to its subfragments.

FIG. 10 depicts an ELISA assay illustrating the binding of Fluorescent tagged SRCRP2 peptide at different concentration with (---) AgI/II of S. mutans and (-) SspB of S. gordonii.

FIG. 11 shows sensorgrams from surface plasmon resonance studies showing the interaction of FL^{AgI/II} and FL^{SspB} at various concentrations with immobilized iSRCR₁, iSRCR₁₂₃ and SAG.

FIG. 12 shows sensorgrams from surface plasmon resonance studies showing the interaction of A₃VP₁^{AgI/II} and A₃VP₁^{SspB} at various concentrations with immobilized iSRCR₁, iSRCR₁₂₃ and SAG.

FIG. 13 shows sensorgrams from surface plasmon resonance studies showing the interaction of C₁₂₃^{AgI/II} and C₁₂₃^{SspB} at various concentrations with immobilized iSRCR₁, iSRCR₁₂₃ and SAG.

FIG. 14 depicts surface plasmon resonance studies illustrating binding of (2 M) Lysozyme but not (2 μM) thaumatin with iSRCR₁ or iSRCR₁₂₃ immobilized on CM5 sensor chip.

FIG. 15 depicts concentration of FL^{AgI/II} and FL^{SspB} bound to immobilized iSRCR₁ and iSRCR₁₂₃ on CM5 sensor chip.

FIG. 16 shows competition experiments with FL^{AgI/II}, A₃VP₁^{AgI/II}, and C₁₂₃^{AgI/II} conducted with immobilized iSRCR₁ (panel A) immobilized iSRCR₁₂₃ (panel B) on Biacore CM5 chip. The direct binding of the fragment prior to competition is shown in bold, followed by the fragments that were tested for their inhibitory activity. Similarly, competition of FL^{SspB}, A₃VP₁^{SspB}, C₁₂₃^{SspB} with immobilized iSRCR₁, iSRCR₁₂₃ and SAG are shown in panels C, D and E. All experiments were carried out in triplicates and the error bars represent standard deviations.

FIG. 17 depicts the binding of 2 μM FL and subfragments of AgI/II of S. mutans in the presence and absence of 2.5 mM CaCl₂ with immobilized (panel A) iSRCR₁ and (panel B) iSRCR₁₂₃ on CM5 sensor chip.

FIG. 18 depicts the binding of 2 μM FL and subfragments of SspB of S. gordonii in the presence and absence of 2.5 mM CaCl₂ with immobilized (panel A) iSRCR₁ and (panel B) iSRCR₁₂₃ on CM5 sensor chip

FIG. 19A shows CD studies demonstrating spectral changes of iSRCR₁ on addition of various concentration of calcium ions (1 mM-100 mM).

FIG. 19B shows CD studies demonstrating spectral changes of iSRCR₁₂₃ on addition of various concentration of calcium ions (1 mM-100 mM).

FIG. 20 depicts DSC showing the stability of iSRCR₁ at various temperature with dose dependent increase of calcium ions.

FIG. 21 depicts Glycoprotein stained SDS-PAGE gel containing iSRCR₁, iSRCR₁₂₃, horse radish peroxidase (HRP, positive control) and soybean trypsin inhibitor (SBTI, negative control).

FIG. 22A shows SPR studies of Galβ1-3-GalNac carbohydrates at different concentrations (0.010 mM-1 mM) with FL^{AgI/II} and FL^{SspB} and its subfragments of AgI/II of S. mutans and SspB of S. gordonii over iSRCR₁ immobilized CM5 sensor chip.

FIG. 22B shows SPR studies of Galβ1-3-GalNac carbohydrates at different concentrations (0.010 mM-1 mM) with FL^{AgI/II} and FL^{SspB} and its subfragments of AgI/II of S. mutans and SspB of S. gordonii over iSRCR₁₂₃ immobilized CM5 sensor chip.

FIG. 23A depicts inhibition studies SRCRP2 peptide at different concentrations (0.005 mM-0.200 mM) with 2 μM FL^{AgI/II} and FL^{SspB} and sub-fragments on iSRCR₁ immobilized CM5 sensor chip using surface plasmon resonance analysis.

FIG. 23B depicts inhibition studies SRCRP2 peptide at different concentrations (0.005 mM-0.200 mM) with 2 μM FL^{AgI/II} and FL^{SspB} and sub-fragments on iSRCR₁₂₃ immobilized CM5 sensor chip using surface plasmon resonance analysis.

FIG. 24 shows the binding of different concentrations (0.250 μM-2 μM) of SRCRs with immobilized iSRCR₁ and iSRCR₁₂₃ on CM5 sensor chip.

FIG. 25 shows models for the adherence of AgI/II to Gp340: (A) Adherence to Gp340 may occur through a single site of AgI/II, (B) AgI/II uses both sites to adhere to an elongated Gp340, (C) Each site on AgI/II adheres to different Gp340 (D) Or both sites on AgI/II adhere to a very large SAG high molecular weight (HMW) complex. SRCRs are shown as medium grey circles, CUB is shown as medium grey squares and ZP is shown as in light grey circles.

FIG. 26 depicts the effect of Galβ1-3GalNac (core-1) carbohydrate on bacterial surface proteins interaction with immobilized Salivary Agglutinin on a CM5 sensor chip.

FIG. 27A depicts a sensorgram from surface plasmon resonance studies showing the interaction of SRCR peptide ETNDANVVARQL (SEQ ID NO:10) with immobilized AgI/II VheI.

FIG. 27B depicts a sensorgram from surface plasmon resonance studies showing the interaction of SRCR peptide ETNDANVVARQL (SEQ ID NO:10) with immobilized SspB VheI.

FIG. 27C depicts a sensorgram from surface plasmon resonance studies showing the interaction of SRCR peptide ETNDANVVARQL (SEQ ID NO:10) with immobilized GbpC.

### DETAILED DESCRIPTION OF THE INVENTION

In the following detailed description, embodiments of the present invention are described in detail to enable practice of the invention. Although the invention is described with reference to these specific embodiments, it should be appreciated that the invention can be embodied in different forms and should not be construed as limited to the embodiments set forth herein. Rather, these embodiments are provided so that this disclosure will be thorough and complete, and will fully convey the scope of the invention to those skilled in the art. All publications cited herein are incorporated by reference in their entireties for their teachings.

The invention includes numerous alternatives, modifications, and equivalents as will become apparent from consideration of the following detailed description.

Unless otherwise defined, all technical terms used herein have the same meaning as commonly understood by one of ordinary skill in the art to which this disclosure belongs.

Also as used herein, the terms “treat,” “treating” or “treatment” may refer to any type of action that imparts a modulating effect, which, for example, can be a beneficial and/or therapeutic effect, to a subject afflicted with a condition, disorder, disease or illness, including, for example, improvement in the condition of the subject (e.g., in one or more symptoms), delay in the progression of the disorder, disease or illness, delay of the onset of the disease, disorder, or illness, and/or change in clinical parameters of the condition, disorder, disease or illness, etc., as would be well known in the art.

As used herein, the terms “prevent,” “preventing” or “prevention of” (and grammatical variations thereof) may refer to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention. In representative embodiments, the term “prevent,” “preventing,” or “prevention of” (and grammatical variations thereof) refer to prevention and/or delay of the onset and/or progression of a metabolic disease in the subject, with or without other signs of clinical disease. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

An “effective amount” or “therapeutically effective amount” may refer to an amount of a compound or composition of this invention that is sufficient to produce a desired effect, which can be a therapeutic and/or beneficial effect. The effective amount will vary with the age, general condition of the subject, the severity of the condition being treated, the particular agent administered, during the duration of the treatment, the nature of any concurrent treatment, the pharmaceutically acceptable carrier used, and like factors within the knowledge and expertise of those skilled in the art. As appropriate, an effective amount or therapeutically effective amount in any individual case can be determined by one of ordinary skill in the art by reference to the pertinent texts and literature and/or by using routine experimentation. (See, for example, Remington, The Science and Practice of Pharmacy (latest edition)).

### Compositions

The present invention is based on the inhibition of the interaction between AgI/II, and/or its homologs, with SAG, more particularly the interaction between AgI/II and the SRCR domains found on Gp340 within the SAG complex, compositions that inhibit this interaction for the prevention and/or inhibition of the formation of dental caries, or the prevention and/or inhibition of the formation of denture plaques, in a subject. In some embodiments of the invention, the inhibitor of the interaction between SAG and AgI/II, and/or its homologs, is a glycan. In one embodiment, the glycan is Galβ1-3-GalNac glycan. In other embodiments of the invention, the inhibitor of the interaction between SAG and AgI/II, and/or its homologs, is a peptide. In some embodiments, the peptide inhibitor of the interaction between AgI/II, and/or its homologs, and SAG, binds to AgI/II, and/or its homologs. In other embodiments, the peptide has sequences identical to or homologous to a scavenger receptor cysteine rich (SRCR) domain from SAG. In one embodiment, the peptide is ETNDANVVARQL (SEQ ID NO:10).

In an embodiment of the invention, provided is a pharmaceutical composition, comprising a therapeutically effective amount of the inhibitor of the interaction between AgI/II, and/or its homologs, with SAG. In other embodiments, the pharmaceutical composition further comprises a pharmaceutically acceptable carrier. The term “pharmaceutically acceptable carrier” as used herein refers to any substance, not itself a therapeutic agent, used as at least in part a vehicle for delivery of a therapeutic agent to a subject. Non-limiting examples of pharmaceutically acceptable components include, without limitation, any of the standard pharmaceutical carriers such as phosphate buffered saline solutions, water, emulsions such as oil/water emulsions or water/oil emulsions, microemulsions, and various types of wetting agents. Further, in preparing such pharmaceutical compositions comprising the active ingredient or ingredients in admixture with components necessary for the formulation of the compositions, other conventional pharmacologically acceptable additives may be incorporated, for example, excipients, stabilizers, wetting agents, emulsifying agents, lubricants, sweetening agents, coloring agents, flavoring agents, isotonicity agents, buffering agents, antioxidants and the like. Additives may include, for example, starch, mannitol, sorbitol, precipitated calcium carbonate, crystalline cellulose, carboxymethylcellulose, dextrin, gelatin, acacia, EDTA, magnesium stearate, talc, hydroxypropylmethylcellulose, sodium metabisulfite, and the like.

Formulations suitable for administering the composition of the present invention may be suitable for oral or buccal (sublingual) administration. The formulation may either be in the form of a solid or a liquid. In some embodiments, forms of formulations suitable for oral administration of the compositions of the present invention include, but are not limited to, a tooth paste or dentifrice composition, an oral hygiene product, for example, an oral hygiene tablet, an oral care composition, for example, an oral rinse, a gel or an additive to a digestible product. Formulations suitable for buccal (sub-lingual) administration include lozenges, tablets, capsules, chewing gum and the like, comprising the active compound, with suitable carriers and additives that would be appreciated by one of skill in the art, for example, binders, diluents, lubricants, disintegrating agents and the like.

Formulations for the prevention of denture plaques may include liquid solutions and/or rinses, either when worn by a subject, or when removed and not being worn by the subject, for example, a solution or rinse for soaking the dentures for a period of time therein.

Liquid formulations include, but are not limited to, solutions, emulsions, dispersions, suspensions and the like with suitable carriers. Additives may include water, alcohols, oils, glycols, preservatives and the like.

In some embodiments, formulations suitable for administering the composition of the present invention may also include additives that may provide greater patient compliance, for example, coloring agents, flavoring agents and the like.

In some other embodiments, the formulations for administering the composition of the present invention may further comprise an additional agent or agents. Such agents may include, but are not limited to, agents for removing plaque, whitening and/or remineralizing teeth, and the like. In still other embodiments, the formulation may further comprise a delivery system, for example, a film or a strip of material, which can be placed against the surface of the teeth of the subject in order to deliver the formulation, for example, as set forth in U.S. Pat. Nos. 5,989,569 and 6,045,811.

### Methods of Administration and Use

Another embodiment of the present invention provides a method for administering to a subject in need thereof a compound or pharmaceutical composition as described herein. For administration, either the compound or pharmaceutical composition is understood as being the active ingredient and capable of administration to a subject, and thus, in some instances, the terms are interchangeable.

Subjects suitable to be treated with the composition, compositions and formulations of the present invention include, but are not limited to mammalian subjects. Mammals according to the present invention include, but are not limited to, canines, felines, bovines, caprines, equines, ovines, porcines, rodents (e.g., rats and mice), lagomorphs, primates, humans and the like, and mammals in utero. Any mammalian subject in need of being treated or desiring treatment according to the present invention is suitable. Human subjects of any gender (for example, male, female or transgender) and at any stage of development (i.e., neonate, infant, juvenile, adolescent, adult, elderly) may be treated according to the present invention.

The method of administration of the compound or pharmaceutical composition as described herein is not particularly limited, and any method that would be appreciated by one of skill in the art for the compound or pharmaceutical composition in a particular formulation as described herein.

### Methods of Identification and Screening

In yet other embodiments of the invention, provided are methods of screening for and identifying inhibitors of the interaction between SAG and AgI/II, and/or homologs thereof, which may be utilized alone or in combination with information on the inhibitors described above to generate still additional inhibitors.

For example, active agents may also be developed by generating a library of molecules, selecting for those molecules which act as ligands for a specified target, and identifying and amplifying the selected ligands. Techniques for constructing and screening combinatorial libraries of oligomeric biomolecules to identify those that specifically bind to a given receptor protein are known. Suitable oligomers include peptides, oligonucleotides, carbohydrates, nonoligonucleotides and nonpeptide polymers. Peptide libraries may be synthesized on solid supports, or expressed on the surface of bacteriophage viruses (phage display libraries). Known screening methods may be used by those skilled in the art to screen combinatorial libraries to identify compounds that antagonize the interaction between SAG and AgI/II, and/or homologs thereof. Techniques are known in the art for screening synthesized molecules to select those with the desired activity, and for labeling the members of the library so that selected active molecules may be identified.

As used herein, “combinatorial library” refers to collections of diverse oligomeric biomolecules of differing sequence, which can be screened simultaneously for activity as a ligand for a particular target. Combinatorial libraries may also be referred to as “shape libraries”, i.e., a population of randomized polymers which are potential ligands. The shape of a molecule refers to those features of a molecule that govern its interactions with other molecules, including Van der Waals, hydrophobic, electrostatic and dynamic.

As noted above, potential active agents or candidate compounds as described can be readily screened for activity in inhibiting the interaction between SAG and AgI/II, and/or a homolog thereof. The method may comprise the steps of: (a) adding or contacting a test compound to an in vitro system comprising SAG and AgI/II, and/or a homolog thereof (this term including binding fragments thereof sufficient to bind to the other); then (b) determining whether the test compound is an inhibitor of the interaction between SAG and AgI/II, and/or homologs thereof; and then (c) identifying the test compound as active or potentially active in inhibiting the formation of dental caries when the test compound is an inhibits the interaction between SAG and AgI/II, and/or homologs thereof. The in vitro system may be in any suitable format that would be appreciated by one of skill in the art. In some embodiments, the in vitro system may be a cell-free system, such as an aqueous preparation of SAG and AgI/II, and/or homologs thereof, or the binding fragments thereof. The contacting, determining and identifying steps may be are carried out in any suitable manner, such as manually, semi-automated, or by a high throughput screening apparatus. The determining step may be carried out by any suitable technique, such as by precipitation, by labeling one of the fragments with a detectable group, all of which may be carried out in accordance with procedures well known to those skilled in the art.

The present invention is more particularly described in the following examples that are intended as illustrative only since numerous modifications and variations therein will be apparent to those skilled in the art.

### Example 1

### Inhibition of SAG Adherence on AgI/II and SspB

### A: Materials and Methods

Expression and Purification of Proteins Used in this Study.

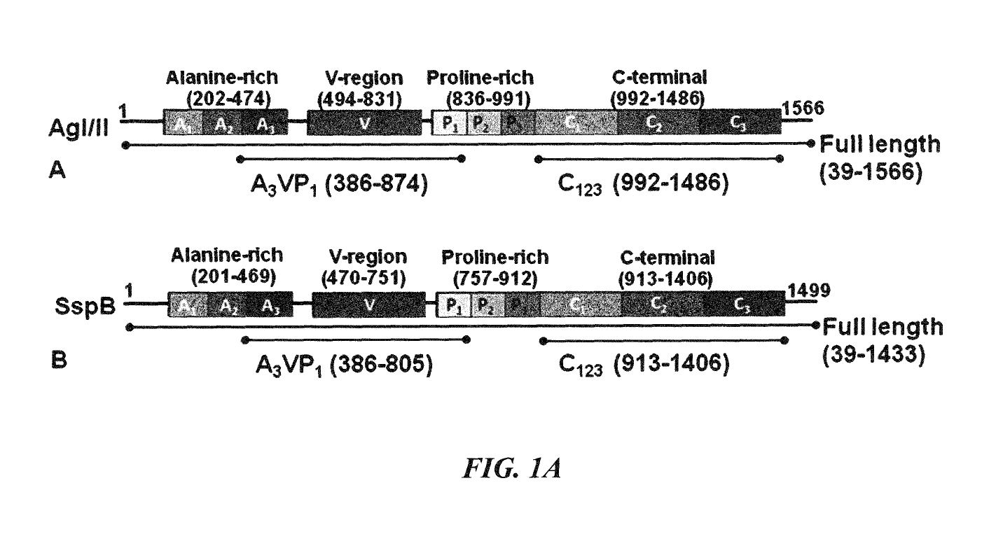

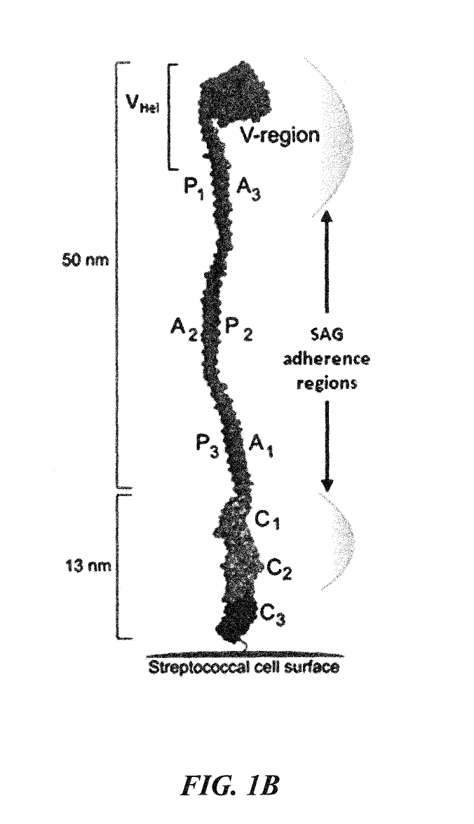

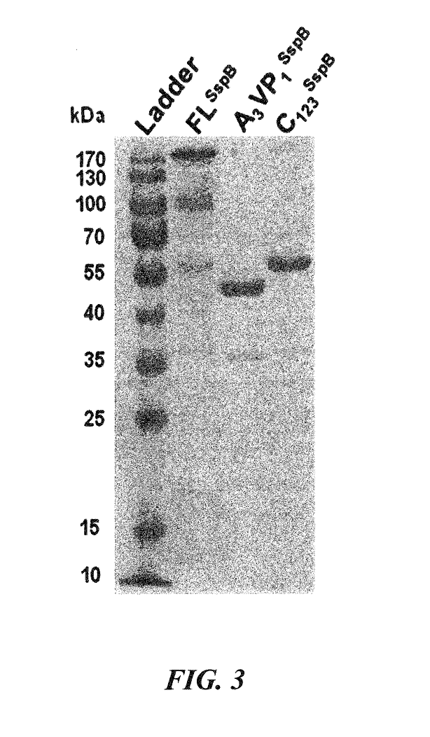

iSRCR₁ and iSRCR₁₂₃ were expressed and purified as described recently by Sangeetha et.al [36], and similarly AgI/II fragments used in this study were prepared as described previously [15]. SspB constructs (FL^{SspB}, A₃VP₁^{SspB} and C₁₂₃^{SspB}), were cloned into pET23d vector (Novagen, Inc) using primers listed in Table 1, restriction enzymes NcoI, NotI, BamHI, and the template plasmid containing the SspB gene FIG. 1. Similar to methods described above for S. mutans AgI/II, the SspB fragments were purified over three columns, HisPrep Nickel affinity, MonoQ and Superdex 200 10/300 GL gel filtration. The purified fragments were analyzed by SDS-PAGE (FIG. 3).

TABLE 1
Primers used for cloning fragments of SspB of Streptococcus gordonii

| Construct | Primers |
|---|---|
| FL^{SsPB} (NcoI) Forward | TATAACCATGGATGAAGTTACAGAGACAACTAGTACAAG (SEQ ID NO: 3) |
| (39-1433) (NotI) Reverse | TTATAGCGGCCGCAGGATCCTTTGGTTTTGGCGTTGG (SEQ ID NO: 4) |
| A₃VP₁^{SsP13} (NcoI) Forward | GCGCCATGGATACCAATGAAGCAGACTACCAA (SEQ ID NO: 5) |
| (386-805) (NotI) Reverse | ATAATTTGCGGCCGCTGGTTTTGATGGCTCCGG (SEQ ID NO: 6) |
| C₁₂₃^{SsPB} (BamHI) Forward | TATAAGGATCCATTTCCACTATAGCAGTTTATTAGC (SEQ ID NO: 7) |
| (913-1406) (XhoI) Reverse | TTATACTCGAGAGATGCATAAGCAACCTTATTAACAG (SEQ ID NO: 8) |

Confocal Microscopy.

S. mutans UA159 and S. gordonii DL1 were grown overnight in TSY (30 g/L of Trypticase soy broth and 0.5 g/L yeast extract, pH 7.2) media on an eight-well Lab Tek Chamber slide system (Sigma). The cells were fixed with 3% paraformaldehyde, washed with binding buffer (20 mM HEPES pH 7.4, 150 mM NaCl and 2.5 mM CaCl₂), and thereafter iSRCR₁ or iSRCR₁₂₃ (10 μM) were added to the cells and incubated for 60 min. The unbound SRCRs were removed by repeated washing using the binding buffer. Subsequently, Alexa fluor 488 conjugated Anti-His-tag antibody (EMD Millipore, Inc) (1:50 dilution) that can bind to the His Tag on SRCRs was added. After 60 min incubation the unbound antibody was washed away thoroughly using binding buffer, and the chamber walls were gently removed. Cover slips were then mounted with 15 μl of fluoromount-G with DAPI (Southern Biotech Inc) to stain bacterial nuclei and were sealed until ready to be imaged. The experiment without SRCRs served as control. All slides were imaged using Leica SP1 UV Confocal Laser Scanning Microscope and Zeiss LSM 710 Confocal Laser Scanning Microscope at the UAB-High resolution imaging facility (UAB-HRIF).

Flow Cytometric Analysis.

S. mutans UA159 and S. gordonii DL1 cells were grown overnight in TSY broth media at 37° C. and washed thoroughly with FACS buffer (20 mM HEPES pH 7.4, 150 mM NaCl and 5% non-fat dry milk) to reduce non-specific binding. Subsequently, 10 μM of iSRCR₁ or iSRCR₁₂₃ were added to 100 μl of cells (1×10⁷ cells/ml) in binding buffer (FACS buffer+2.5 mM CaCl₂) and incubated for 60 min at 37° C. The cells were later washed thoroughly with binding buffer to remove unbound iSRCRs. Subsequently, Anti-His tag Alexafluor 488 antibody (1:50 dilution) (EMD Millipore, Inc) that adheres to the His tag present on SRCRs was added to the cells and incubated for 30 min, washed three times and resuspended in 200 μl of binding buffer. Samples without iSRCRs served as control. Samples were then assayed using the FACScan machine (BD Biosciences) at the Analytical and Preparative Cytometry Facility (APCF) at UAB, and data obtained was analyzed using FlowJo 7.2.4 software.

Aggregation Assays.

Aggregation assays were performed as described earlier [37] with slight modifications. Briefly, S. mutans UA159 and S. gordonii DL1 cells were grown in TSY broth media overnight at 37° C. in the presence of 5% CO₂. The bacteria were centrifuged at 5000×g and washed with a buffer containing 20 mM HEPES pH 7.4, 150 mM NaCl and re-suspended to an approximate OD₇₀₀ of 1. The bacterial suspension (900 μl) was mixed with 100 μl of SAG or iSRCR₁ (10 μM) or iSRCR₁₂₃ (10 M) or commercially synthesized (Think Peptides, Inc) SRCRP2 peptide with fluorescein amidite (FAM) at the carboxyl end (QGRVEVLYRGSWGTVCK-[FAM]) (SEQ ID NO:9, at both 400 μg/ml and 1 mg/ml) in the presence of 6 mM CaCl₂ and aggregation was measured by recording OD₇₀₀over 60 min at 5 min intervals, where the buffer alone was used as control. All experiments were carried out at least five times, and the results were analyzed with One-way ANOVA. Post-hoc testing where *, P<0.05 was considered statistically significant and results were presented as the percentage of cells aggregated.

ELISA.

The binding between SRCRs and oral streptococci fragments were analyzed using ELISA. Both iSRCR₁ and iSRCR₁₂₃ (10 g/well) in carbonate-bicarbonate buffer (pH 9.6) were coated on a black ELISA plate individually, washed with binding buffer containing 20 mM HEPES, pH 7.5, 150 mM NaCl and 2.5 mM CaCl₂ (pH 7.2) and blocked with 3% non-fat dry skim milk. Various concentrations of Alexafluor 488 conjugated Anti-His-tag antibody (Millipore, Inc) ranging from 10 jag/ml to 0.1 ng/ml was added to 10 g of each of the FL or A₃VP₁ or C₁₂₃ fragments of S. mutans or S. gordonii for 3 hours at room temperature and were dialyzed into the binding buffer. Two hundred microliters of the fluorescently labeled fragments of AgI/II and SspB were added to the wells containing immobilized iSRCRs and incubated for 3 hours. SRCR immobilized wells without fluorescently labeled analytes were used as controls. Later these wells were washed three times with binding buffer and data were recorded at an excitation wavelength of 495 nm with the emission at 519 nm using Synergy 2-multimode microplate reader (Synergy, Inc). The assays were performed in triplicates and thereafter results were analyzed.

The binding between synthesized SRCRP2 peptide and SRCR were analyzed utilizing the FAM label on the SRCRP2 peptide. Briefly, both iSRCR₁ and iSRCR₁₂₃ (10 μg/well) in carbonate-bicarbonate buffer (pH 9.6) were coated on a black ELISA plate individually, washed with binding buffer containing 20 mM HEPES, pH 7.5, 150 mM NaCl and 2.5 mM CaCl₂ (pH 7.2) and blocked with 3% non-fat dry skim milk. Serial dilution of the SRCRP2 peptide (200 μl) ranging from 3 ng/ml to 0.001 mg/ml in binding buffer were incubated with SRCRs for 3 hours at room temperature. SRCR immobilized wells without fluorescently labeled SRCRP2 peptides were used as controls. Later the wells were washed with binding buffer and the data was recorded at an excitation wavelength of 495 nm with the emission at 519 nm using Synergy 2-multimode microplate reader (Synergy, Inc).

Surface Plasmon Resonance.

Real time binding analyses of the SRCR domains with AgI/II fragments were carried out using the BIAcore 2000 system. The CM5 chip was labeled with ligands iSRCR₁ or iSRCR₁₂₃ SAG (a gift from Dr. Jeannine Brady, University of Florida, Gainesville, prepared as previously described [37,38], using the amine coupling kit (GE healthcare, Inc). The control and experimental surfaces were blocked using 1 M ethanolamine. Various concentrations of analytes (0.125 M to 2 μM) of S. mutans AgI/II or S. gordonii SspB fragments and (Table 2) 2 M of Lysozyme (Positive control) and Thaumatin (Negative control) were injected over the prepared chip surfaces and dissociation were measured for 8-10 minutes at a flow rate of 20 μl/min of binding buffer (20 mM HEPES, pH 7.4, 150 mM NaCl, 2.5 mM CaCl₂) at 25° C. Self association of iSRCR1 or iSRCR₁₂₃ (at 2 μM) were also determined in a similar manner as described above. Between these experiments the regeneration of the surface was accomplished using solutions as indicated in Table 2. Finally to determine the effect of calcium SPR analysis was carried out by dialyzing the analytes and ligands in binding buffer devoid of CaCl₂.

On-chip Deglycosylation of the iSRCR₁ and iSRCR₁₂₃ was carried out after immobilizing them on CM5 sensor chip. Enzymatic deglycosylation was done to remove N- and O-linked carbohydrates from iSRCR₁ and iSRCR₁₂₃. Briefly, after immobilization of iSRCR₁ and iSRCR₁₂₃, deglycosylation was carried out at native conditions by incubating the entire chip surface externally with a cocktail containing a total reaction volume of 40 μl made up of 4 μl of 10×G7reaction buffer, 4 μl of 10% NP40, 4 μl of Neuraminidase (Sigma), 18 μl water and 10 μl of O-glycosidase (New England Biolabs, Inc.,) and similarly following manufacturers protocols for EndoH (New England Biolabs, Inc). Later, the chip was sealed and incubated overnight at 37° C. Subsequently the chip was thoroughly washed with binding buffer (20 mM HEPES, 150 mM NaCl, 2.5 mM CaCl₂) to remove the deglycosylating enzymes and other remnants. Binding studies with FL^{AgI/II} and FL^{SspB} and subfragments were then carried out as described above and regenerated as described in Table 2.

The utilization of a bivalent adherence model to elucidate the kinetics had inherent difficulties in clearly distinguishing affinities for each region, particularly for FL^{AgI/II} and FL^{SspB}. In addition, the SRCR holding two distinct surfaces compounded the elucidation of individual kinetics, and presently there are no modeling protocols available to determine the individual affinities for such a system, therefore for simplicity we have utilized a single site 1:1 Langmuir model. All experiments were carried out in triplicates and the kinetics of the association (K_{A}) and dissociation (K_{D}) rate constants were deduced using the 1:1 Langmuir Kinetic model on the BIA-Evaluation software [39]. The concentration (C in μM) of analyte (FL^{AgI/II} or FL^{SspB} at 2 μM) that adhered to the immobilized ligand (iSRCR₁, iSRCR₁₂₃) within the flow cell was calculated using the formula C=(RU/MW)×(1/V), where RU is resonance unit (1 RU=1 pg of bound protein), MW is molecular weight of analyte and V is volume of flow cell (1.2×10⁻¹⁰ L).

TABLE 2
Concentration of analytes in surface plasmon resonance studies

| | | | | | Regeneration | Regeneration |
|---|---|---|---|---|---|---|
| | Ligand | | | | Buffer for | Buffer for |
| Analyte | Ligand 1 | Ligand 2 | Ligand 3 | Running Buffer | Ligands 1 & 2 | Ligand 3 |
| FL^{AgI/II} | iSRCR₁ | iSRCR₁₂₃ | SAG | 20 mM HEPES, pH | 1M NaCl, | 10 mM HCl |
| | | | | 8.0, 150 mm NaCl, | 20 mM EDTA | |
| | | | | 2.5 mM CaCl₂ | pH 7.2 | |
| A₃VP₁^{AgI/II} | iSRCR₁ | iSRCR₁₂₃ | SAG | 20 mM HEPES, pH | 1M NaCl, | 10 mM HCl |
| | | | | 8.0, 150 mm NaCl, | 20 mM EDTA | |
| | | | | 2.5 mM CaCl₂ | pH 7.2 | |
| C₁₂₃^{AgI/II} | iSRCR₁ | iSRCR₁₂₃ | SAG | 20 mM HEPES, pH | 1M NaCl, | 10 mM HCl |
| | | | | 8.0, 150 mm NaCl, | 20 mM EDTA | |
| | | | | 2.5 mM CaCl₂ | pH 7.2 | |
| FL^{SspB} | iSRCR₁ | iSRCR₁₂₃ | SAG | 20 mM HEPES, pH | 1M NaCl, | 10 mM HCl |
| | | | | 8.0, 150 mm NaCl, | 20 mM EDTA | |
| | | | | 2.5 mM CaCl₂ | pH 7.2 | |
| A₃VP₁^{SspB} | iSRCR₁ | iSRCR₁₂₃ | SAG | 20 mM HEPES, pH | 1M NaCl, | 10 mM HCl |
| | | | | 8.0, 150 mm NaCl, | 20 mM EDTA | |
| | | | | 2.5 mM CaCl₂ | pH 7.2 | |
| C₁₂₃^{SspB} | iSRCR₁ | iSRCR₁₂₃ | SAG | 20 mM HEPES, pH | 1M NaCl, | 10 mM HCl |
| | | | | 8.0, 150 mm NaCl, | 20 mM EDTA | |
| | | | | 2.5 mM CaCl₂ | pH 7.2 | |
| Thaumatin | iSRCR₁ | iSRCR₁₂₃ | SAG | 20 mM HEPES, pH | 1M NaCl, | 10 mM HCl |
| (negative | | | | 8.0, 150 mm NaCl, | 20 mM EDTA | |
| control) | | | | 2.5 mM CaCl₂ | pH 7.2 | |
| Lysozyme | iSRCR₁ | iSRCR₁₂₃ | SAG | 20 mM HEPES, pH | 1M NaCl, | 10 mM HCl |
| (positive | | | | 8.0, 150 mm NaCl, | 20 mM EDTA | |
| control) | | | | 2.5 mM CaCl₂ | pH 7.2 | |
| iSRCR₁ | iSRCR₁ | iSRCR₁₂₃ | | 20 mM HEPES, pH | 10 mM HCl | 10 mM HCl |
| | | | | 8.0, 150 mm NaCl, | | |
| | | | | 2.5 mM CaCl₂ | | |
| iSRCR₁₂₃ | iSRCR₁ | iSRCR₁₂₃ | | 20 mM HEPES, pH | 10 mM HCl | 10 mM HCl |
| | | | | 8.0, 150 mm NaCl, | | |
| | | | | 2.5 mM CaCl₂ | | |

Competition Adherence Assays.

To determine whether AgI/II domains bound to the same site on SRCR domains, competitive binding SPR experiments were conducted in triplicates as previously described [16] where each fragment of AgI/II (FL, A₃VP₁, or C₁₂₃) or SspB (FL, A₃VP₁ or C₁₂₃) was initially passed over the chip surface immobilized with either iSRCR₁, iSRCR₁₂₃ or SAG for 60 seconds to saturate available binding sites. The response curve of AgI/II or SspB first fragment was recorded, where the maximal RU (RU₁) was considered as the base line for the second injection, and thereafter the competing fragment was injected and its response was recorded as RU₂. The adherence of the second fragment was then calculated (RU₂−RU₁) for all SPR competing assay as reported earlier [16].

Circular Dichroism.

Spectroscopic studies were carried out on an Olis DSM 100 circular dichroism spectrophotometer with 0.2 mm path length quartz cell. Recombinant iSRCR₁ or iSRCR₁₂₃ at concentration of 1 mg/ml in a buffer containing 20 mM HEPES pH 7.4, 150 mM NaCl and 2.5 mM CaCl₂ at 22° C. were scanned between 200-260 nm and the spectra was recorded (10 times). Similarly, the conformational changes of SRCRs on addition of different concentrations of calcium (2, 4, 6, 8 and 10 mM) in binding buffer as well as SRCR samples devoid of calcium (control) were analyzed by scanning the spectra between 200-260 nm for nearly 10 times. Using CONTIN/LL algorithm implemented in CDPRO [40] the protein secondary structure were assigned.

Differential Scanning Calorimetry.

The thermostability of SRCRs in the presence of calcium ions was analyzed using Microcal MC-II differential scanning calorimeter (GE HealthCare, USA) as described earlier [41]. Briefly, iSRCR₁ or iSRCR₁₂₃ at concentration of 1 mg/ml was mixed and incubated with different concentration of CaCl₂ ranging from (0-100 mM) to final volume of 400 μl of buffer containing 20 mM HEPES, 150 mM NaCl), and buffer without SRCRs served as control. Data were recorded with calorimetric scanning rates that ranged from 30° C./h to 90° C./hat 30 psi pressure. The data collected was analyzed for the unfolding temperature (Tₜ), and the calorimetric (ΔH_{cal}) and van't Hoff (ΔHᵥ) unfolding enthalpies using the Origin software package (MicroCal).

Glycoprotein Staining and GC-MS Analysis of SRCRs Carbohydrates.

The SRCR₁ and SRCR₁₂₃ proteins were electrophoretically separated on a 12.5% SDS-PAGE gel and stained by glycoprotein staining kit (Pierce, Inc), where Horse radish peroxidase (HRP) and Soybean trypsin Inhibitor (SBTI) were used as positive and negative control respectively. The glycosyl composition analysis of purified iSRCR₁ and iSRCR₁₂₃ were done by the preparation and gas chromatograph-mass spectrometry (GC-MS) of trimethylsilyl (TMS) methyl glycosides as previously described [42].

Adherence/Inhibition Studies.

Carbohydrates Galβ1-3-GalNac and Mannose (identified from glycan profile analysis) at different concentrations (0.010, 0.050, 0.1, 0.5 and 1 mM) were incubated with 2 μM of each FL, A₃VP₁ and C₁₂₃ of AgI/II and SspB and the interaction with immobilized iSRCR₁ and iSRCR₁₂₃ with running buffer containing 20 mM HEPES, 150 mM NaCl and 2.5 mM CaCl₂, pH 7.4 at 25° C. and with flow rate of 20 μl/min was analyzed. Direct adherence of these carbohydrates alone served as the control and all calculations were carried out using the BIAevaluation software. Using the same protocol above at varying concentrations the effect of SRCRP2 peptide on the adherence inhibition was assessed.

Analytical Ultracentrifugation.

iSRCR₁ or iSRCR₁₂₃ (0.5 mg/ml) in a buffer containing 20 mM Tris pH 8.0, 150 mM NaCl, and 1 mM EDTA were subjected to sedimentation velocity experiments on a Beckman Optima XL-A as previously described [15]. Briefly, the samples were centrifuged to 45,000 rpm with the temperature maintained at 20° C., and where absorbance at 280 nm across the cell recorded every 5 min. Using Sednterp, buffer density values of 1.0052 g/ml, protein partial specific volumes of 0.720 and 0.714 g/ml, and hydration values of 0.365 and 0.370 g/g for iSRCR₁ and iSRCR₁₂₃, respectively were calculated [43,44].

### B: Results

AgI/II and SspB Constructs.

Constructs iSRCR₁ (15 kDa) and iSRCR₁₂₃ (43 kDa) were prepared as described earlier [36]. S. mutans AgI/II constructs FL^{AgI/II} (167.5 kDa, earlier referred to as CG14), A₃VP₁^{AgI/II} (54.4 kDa) and C₁₂₃^{AgI/II} (57.3 kDa) were expressed and purified as described earlier [15,16]. The equivalent constructs for S. gordonii SspB, FL^{SspB} (151.9 kDa), A₃VP₁^{SspB} (46.3 kDa) and C₁₂₃^{SspB} (56.2 kDa) were cloned, expressed and purified for this study as described in the materials and methods section. The purity of proteins was qualitatively assessed to be >95% from SDS-PAGE gels (FIG. 3).

Confocal Microscopy.

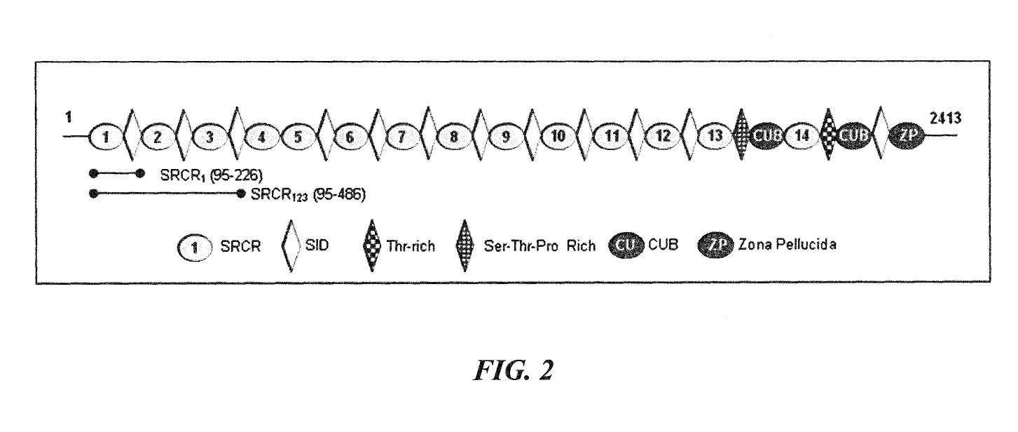

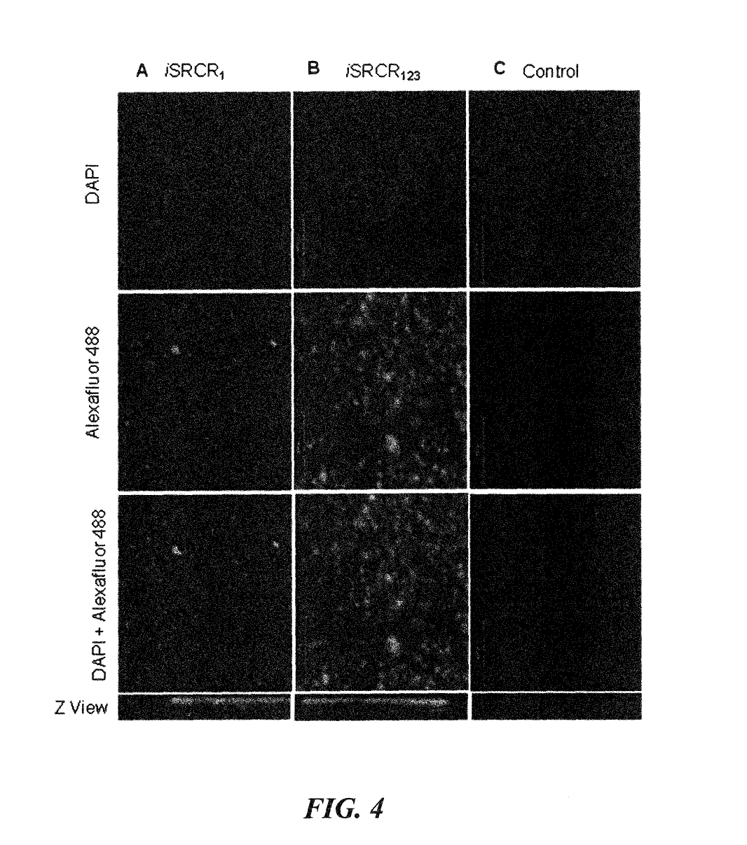

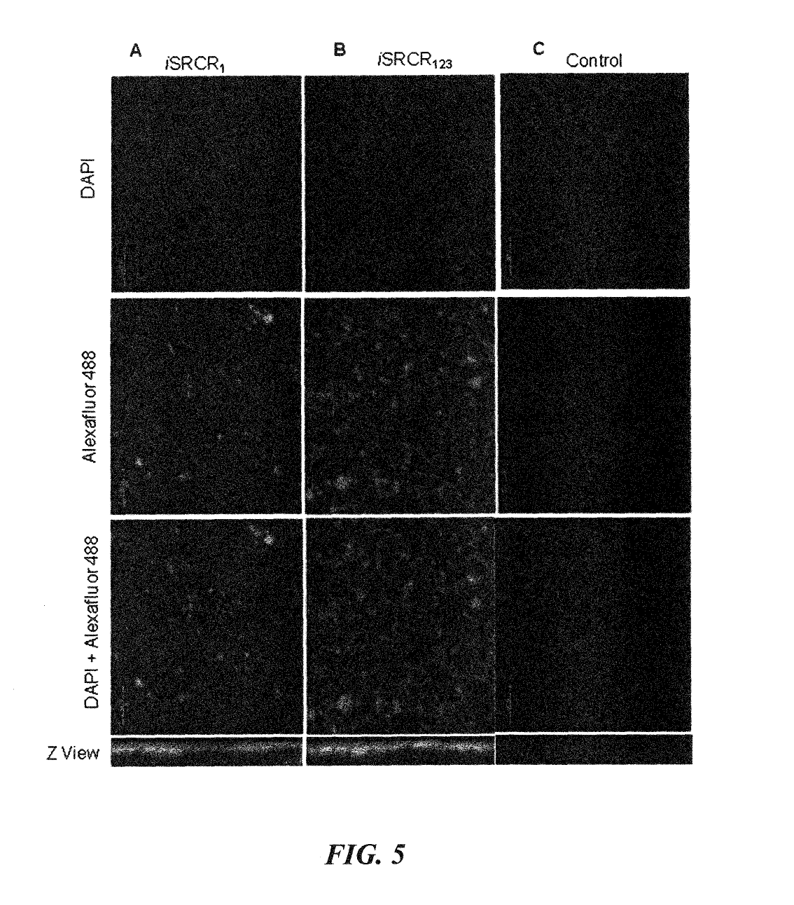

Adherence of iSRCRs to S. mutans and S. gordonii cells were qualitatively analyzed using confocal microscopy. In FIGS. 1 and 2 bacterial nuclei stained with DAPI are displayed in blue, and in green the Anti-His tag Fluor 488 antibody that recognizes the His Tag on the SRCRs. The controls in the absence of SRCRs were only stained by DAPI (FIG. 4, panel A), whereas green fluorescence observed (FIG. 4, panel B and C) confirms the binding of iSRCR₁ and iSRCR₁₂₃ domains to S. mutans cells. Similar adherence was observed with S. gordonii cells (FIG. 5, panels A-C). These findings indicate that iSRCRs interact with both S. mutans and S. gordonii, and that the iSRCR₁ adheres poorly compared to iSRCR₁₂₃, thus indicating that multiple SRCR domains have better adherence capability compared to that of a single SRCR domain. Z view of the confocal microscopy picture clearly shows that the SRCRs adhere only to the top surface of immobilized S. mutans and S. gordonii cells and not to the plate.

Flow Cytometry.

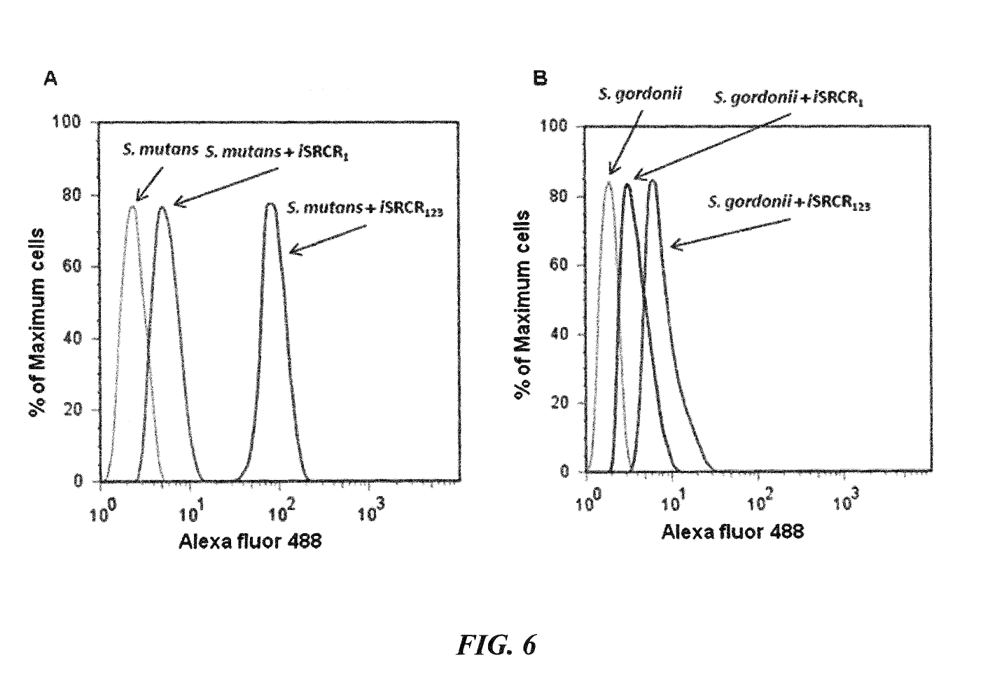

From representative histograms, iSRCR₁₂₃ shows nearly a 100 fold increase in fluorescence intensity compared to the control, whereas iSRCR₁ displays only a 10 fold increase (FIG. 6, panel A). In the case of S. gordonii, iSRCR₁ and iSRCR₁₂₃ display lower adherence (FIG. 6, panel B), not as profoundly as evidenced with S. mutans. Taken together, iSRCR₁₂₃ definitively adheres better than iSRCR₁ again indicating that multiple domains play a co-operative role in this interaction. Furthermore, these results also demonstrate that S. mutans has more pronounced adherence to iSRCR₁₂₃ compared to S. gordonii.

Aggregation Assays.

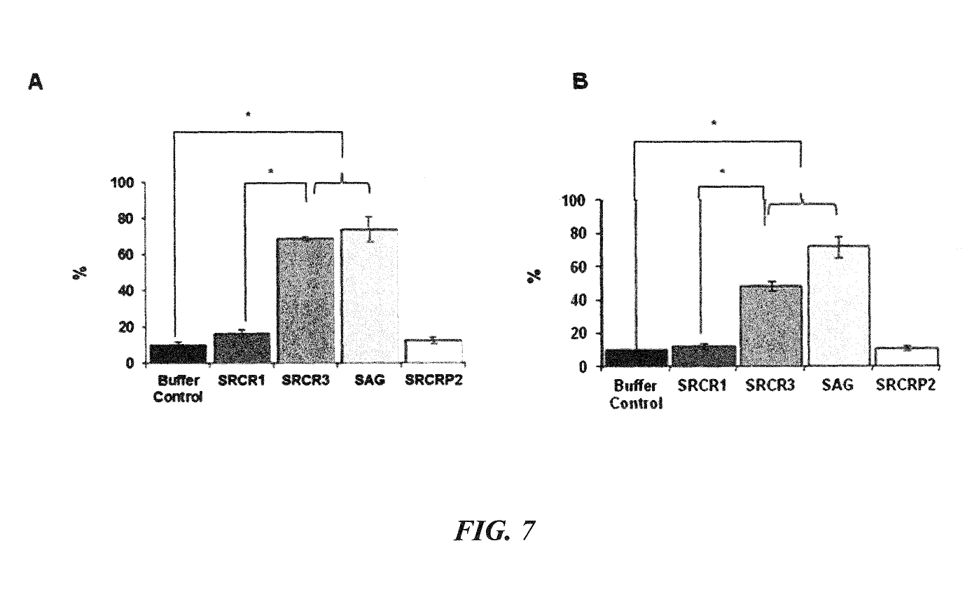

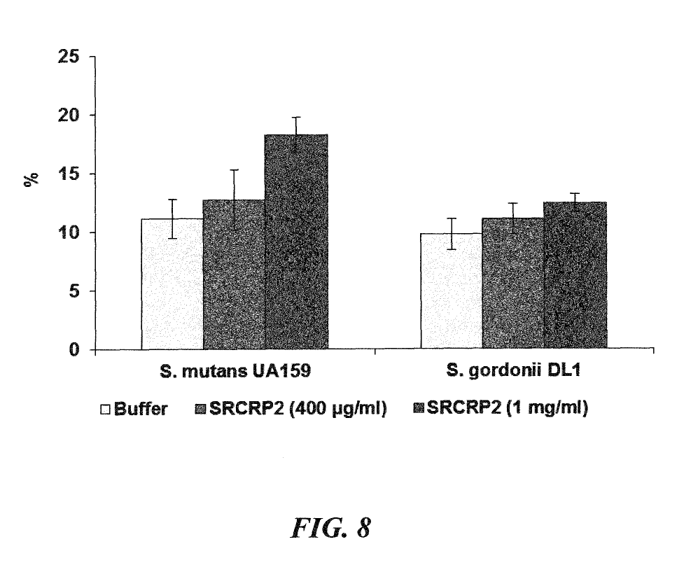

SAG has been well documented to have aggregation properties particularly with S. mutans and S. gordonii [35,45,46]. In this regard, we tested whether individual SRCR domains possess aggregation property as that of SAG. In the presence of iSRCR₁₂₃, 69% of S. mutans and 48% of S. gordonii aggregated while iSRCR₁ aggregated 17% of S. mutans and 13% of S. gordonii (FIG. 7, panels A and B). The positive control SAG aggregated S. mutans by 74% and S. gordonii by 72%. Earlier studies with consensus peptide, SRCRP2 derived from SRCR domains, aggregated a variety of bacteria [20,34] but however in our current study compared to iSRCR₁ and iSRCR₁₂₃, the SRCRP2 peptide displayed limited aggregation with S. mutans (12%) and S. gordonii (11%). The SRCRP2 peptide even at higher concentrations like 1 mg/ml was able to aggregate S. mutans (18%) and S. gordonii (12%) only minimally (FIG. 8).

ELISA.

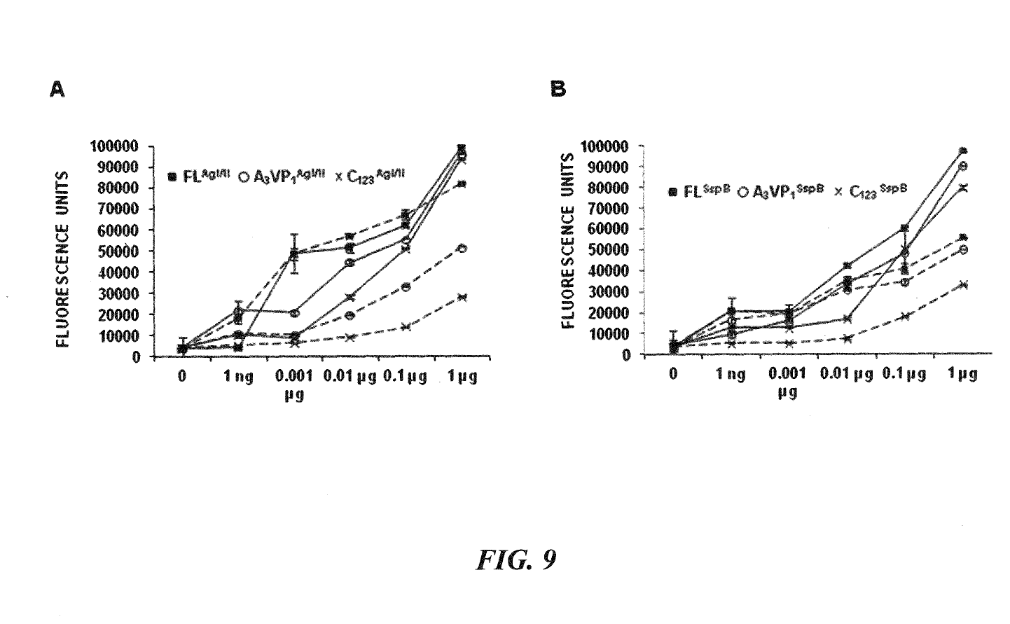

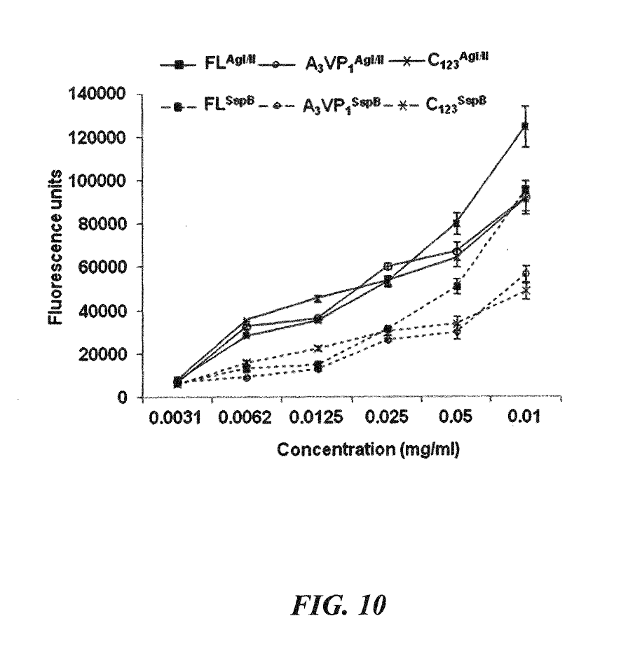

Both FL^{AgI/II} and FL^{SspB} displayed better adherence to iSRCR₁ and iSRCR₁₂₃compared to their sub fragments A₃VP₁ and C₁₂₃ (FIG. 9, panels A and B). Similarly, the SRCRP2 peptide bound to FL^{AgI/II} and FL^{SspB} with higher affinity compared to their sub fragments A₃VP₁ and C₁₂₃ of AgI/II or SspB (FIG. 10).

Adherence Assays and Quantitation.

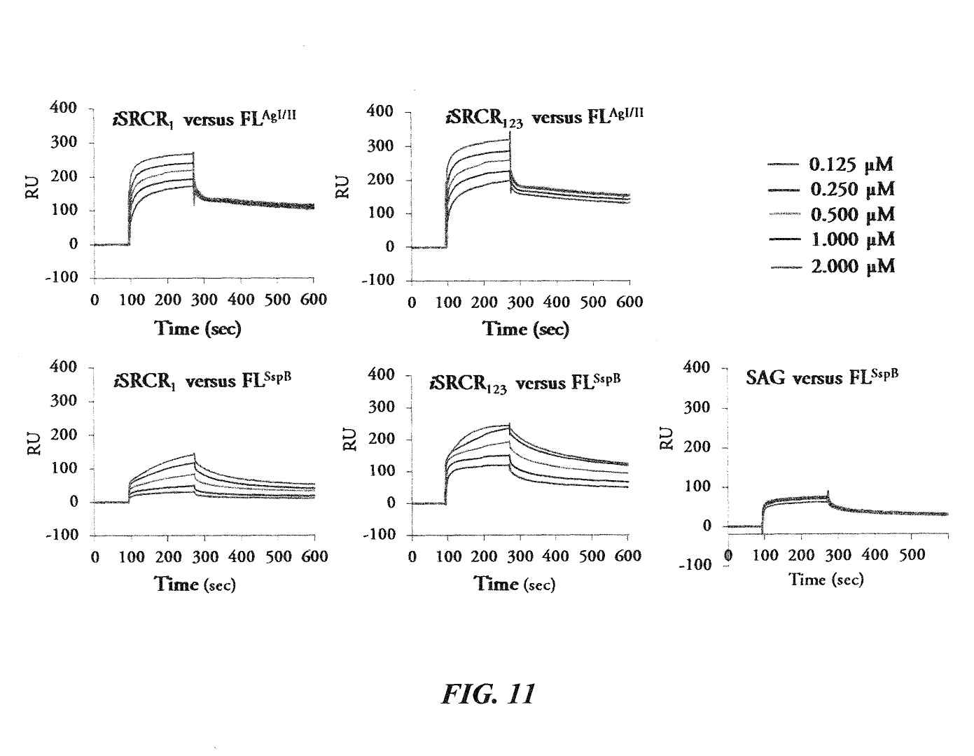

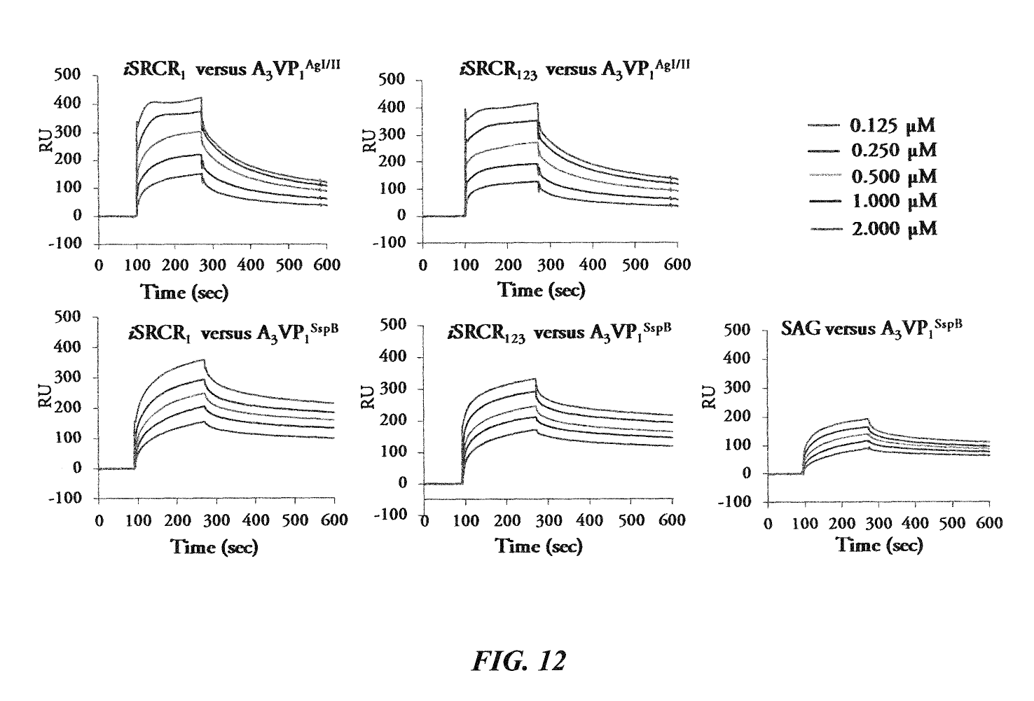

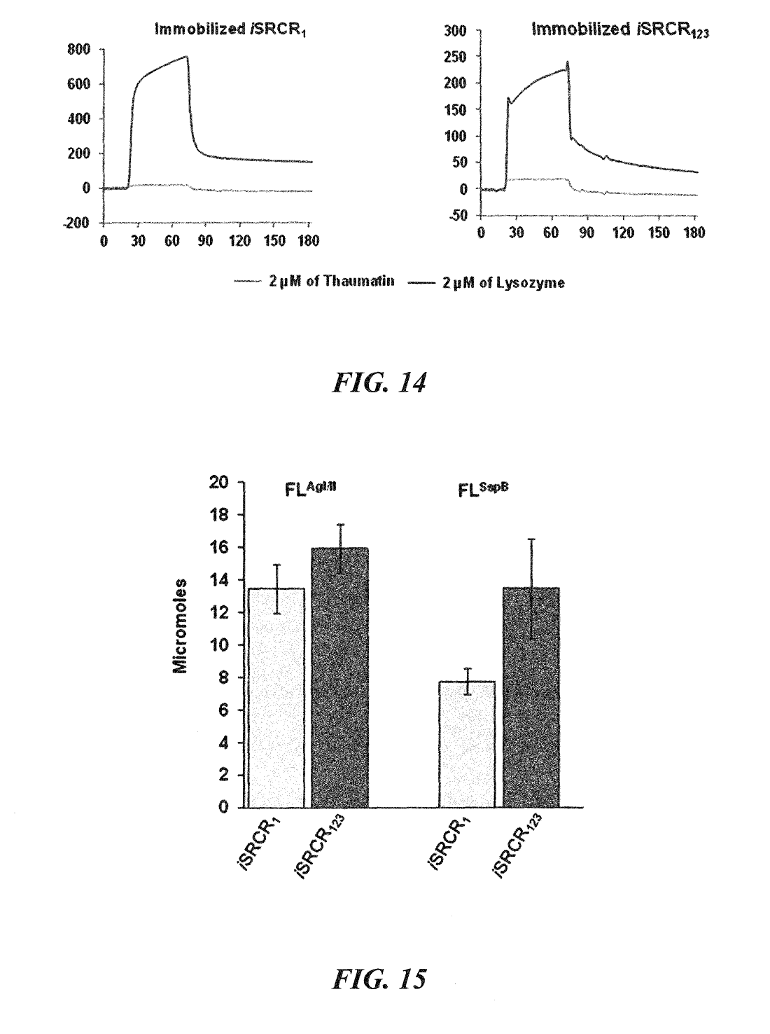

SPR was used to quantify the affinities between immobilized iSRCRs and the analytes FL, A₃VP₁ and C₁₂₃ of AgI/II and SspB (Table 3; FIGS. 11-13). The adherence to lysozyme (positive control) and not to thaumatin (negative control) confirmed the specificity of the SRCR domains (FIG. 14). The interaction of the FL^{AgI/II} with SAG is one order of magnitude lower (3.33×10⁻⁸ M [15]) than that of the FL^{SspB}(6.15×10′⁹ M), and indicates that SspB adheres with higher affinity to SAG. While FL^{SspB}interacts with higher affinity to SAG, the reverse is observed with the individual SRCR domains, where FL^{AgI/II} displays a higher affinity with both iSRCR₁ (7.69×10⁻⁹ M vs 2.56×10⁻⁷ M) and iSRCR₁₂₃ (7.46×10⁻⁹ M vs 4.21×10⁻⁸ M). In all other cases, the FL^{AgI/II} and FL^{SspB} had similar or higher affinities compared to the individual fragments except for A₃VP₁^{SspB} which displays one order higher affinity (3.41×10⁻⁸ M) with iSRCR₁. Interestingly, C₁₂₃ of AgI/II and SspB, which is located near the streptococcal cell wall (FIG. 1) displayed similar affinities to the iSRCRs (1.51×10⁻⁷ M and 4.64×10⁻⁷ M with iSRCR₁ and 8.70×10⁻⁸ M and 6.21×10⁻⁷ M with iSRCR₁₂₃). These affinities indicate that the binding mechanism adopted by the A₃VP₁ region at the apex of the molecule may vary between species. Overall, the similarity in the affinities observed between all AgI/II and SspB fragments with both iSRCR₁, iSRCR₁₂₃ and SAG, strongly proves that a single SRCR domain contains the adherence sites for AgI/II and SspB. Although FL^{AgI/II} and FL^{SspB} displayed similar affinities, the quantity of protein that adhered to iSRCR₁₂₃ was 16% and 43% higher than iSRCR₁ (FIG. 15), and this result is in conjunction with our earlier whole cell assays, where iSRCR₁₂₃ displayed better adherence and aggregation.

TABLE 3
Surface plasmon resonance studies

| Ligand | Analyte | kₐ (1/Ms) | k_{d} (1/s) | K_{A} (1/M) | K_{D} (M) |
|---|---|---|---|---|---|
| iSRCR₁ | FL^{AgI/II} | 1.27 × 10⁵ | 9.76 × 10⁻⁴ | 1.31 × 10⁸ | 7.69 × 10⁻⁹ |
| | A₃VP₁^{AgI/II} | 2.80 × 10⁴ | 3.27 × 10⁻³ | 8.57 × 10⁶ | 1.17 × 10⁻⁷ |
| | C₁₂₃^{AgI/II} | 1.54 × 10⁴ | 2.32 × 10⁻³ | 6.46 × 10⁶ | 1.51 × 10⁻⁷ |
| iSRCR₁ | FL^{AgI/II} | 4.97 × 10⁴ | 57.5 × 10⁻³ | 8.65 × 10⁵ | 1.16 × 10⁻⁶ |
| (Deglycosylated) | A₃VP₁^{AgI/II} | 5.27 × 10³ | 2.74 × 10⁻³ | 1.92 × 10⁶ | 5.20 × 10⁻⁷ |
| | C₁₂₃^{AgI/II} | 1.18 × 10³ | 24.6 × 10⁻³ | 4.81 × 10⁴ | 2.08 × 10⁻⁵ |
| iSRCR₁₂₃ | FL^{AgI/II} | 9.97 × 10⁴ | 7.43 × 10⁻⁴ | 1.34 × 10⁸ | 7.46 × 10⁻⁹ |
| | A₃VP₁^{AgI/II} | 1.99 × 10⁴ | 2.49 × 10⁻³ | 7.98 × 10⁶ | 1.25 × 10⁻⁷ |
| | C₁₂₃^{AgI/II} | 8.29 × 10³ | 7.21 × 10⁻⁴ | 1.15 × 10⁷ | 8.70 × 10⁻⁸ |
| iSRCR₁₂₃ | FL^{AgI/II} | 3.16 × 10³ | 4.64 × 10⁻⁴ | 6.82 × 10⁶ | 1.47 × 10⁻⁷ |
| (Deglycosylated) | A₃VP₁^{AgI/II} | 4.07 × 10³ | 3.33 × 10⁻³ | 1.22 × 10⁶ | 8.18 × 10⁻⁷ |
| | C₁₂₃^{AgI/II} | 3.01 × 10³ | 6.58 × 10⁻³ | 4.58 × 10⁵ | 2.18 × 10⁻⁶ |
| iSRCR₁ | FL^{SspB} | 9.25 × 10³ | 2.37 × 10⁻³ | 3.91 × 10⁶ | 2.56 × 10⁻⁷ |
| | A₃VP₁^{SspB} | 2.85 × 10⁴ | 9.53 × 10⁻⁴ | 2.99 × 10⁷ | 3.41 × 10⁻⁸ |
| | C₁₂₃^{SspB} | 1.07 × 10⁴ | 4.98 × 10⁻³ | 2.16 × 10⁶ | 4.64 × 10⁻⁷ |
| iSRCR₁ | FL^{SspB} | 8.30 × 10³ | 1.04 × 10⁻³ | 7.97 × 10⁶ | 1.25 × 10⁻⁷ |
| (Deglycosylated) | A₃VP₁^{SspB} | 3.70 × 10⁴ | 12.50 × 10⁻³ | 2.96 × 10⁶ | 3.38 × 10⁻⁷ |
| | C₁₂₃^{SspB} | 1.90 × 10⁴ | 20.7 × 10⁻³ | 9.21 × 10⁵ | 1.09 × 10⁻⁶ |
| iSRCR₁₂₃ | FL^{SspB} | 1.82 × 10⁴ | 7.59 × 10⁻⁴ | 2.38 × 10⁷ | 4.21 × 10⁻⁸ |
| | A₃VP₁^{SspB} | 3.85 × 10⁴ | 6.97 × 10⁻⁴ | 5.53 × 10⁷ | 1.81 × 10⁻⁸ |
| | C₁₂₃^{SspB} | 6.62 × 10³ | 4.11 × 10⁻³ | 1.61 × 10⁶ | 6.21 × 10⁻⁷ |
| iSRCR₁₂₃ | FL^{SspB} | 6.92 × 10³ | 1.32 × 10⁻³ | 5.24 × 10⁶ | 1.91 × 10⁻⁷ |
| (Deglycosylated) | A₃VP₁^{SspB} | 7.80 × 10⁴ | 56.7 × 10⁻³ | 1.38 × 10⁶ | 7.27 × 10⁻⁷ |
| | C₁₂₃^{SspB} | 2.42 × 10³ | 15.1 × 10⁻³ | 1.61 × 10⁵ | 6.22 × 10⁻⁶ |
| SAG | FL^{SspB} | 3.20 × 10⁵ | 1.97 × 10⁻³ | 1.63 × 10⁸ | 6.15 × 10⁻⁹ |
| | A₃VP₁^{SspB} | 3.71 × 10⁴ | 1.17 × 10⁻³ | 3.19 × 10⁷ | 3.14 × 10⁻⁸ |
| | C₁₂₃^{SspB} | 2.97 × 10⁴ | 2.77 × 10⁻³ | 1.07 × 10⁷ | 9.34 × 10⁻⁸ |

Competitive Binding Experiments.

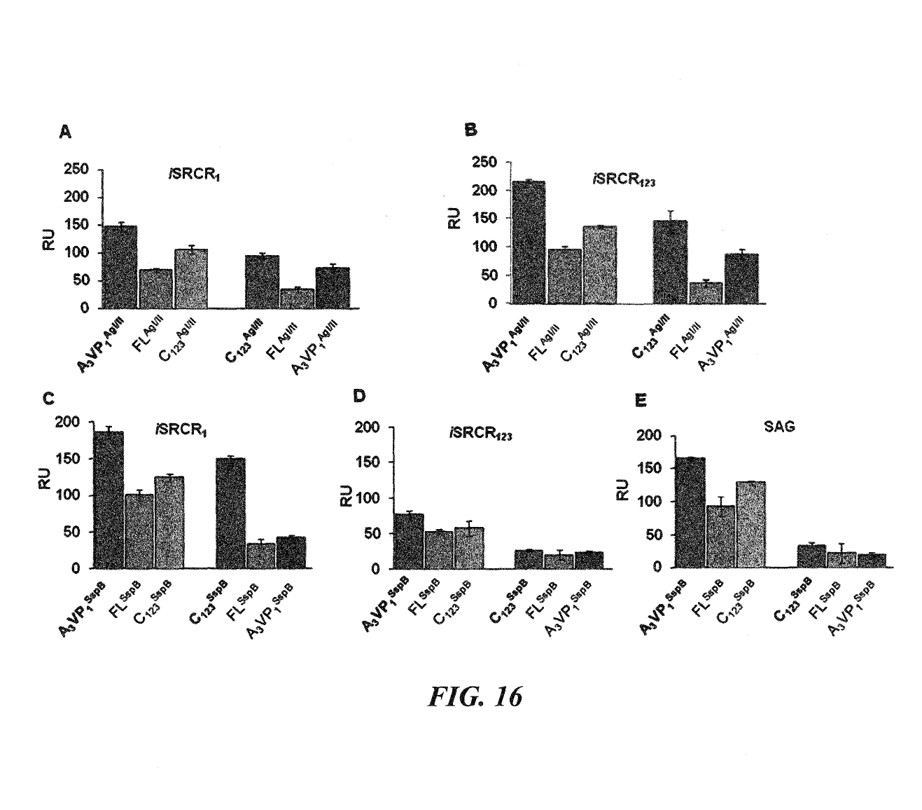

We previously demonstrated that the interaction of AgI/II with SAG was multivalent, where A₃VP₁^{AgI/II} as well as C₁₂₃^{AgI/II} interacted with two distinct surfaces on SAG [16]. To determine if the individual SRCR domains contain these distinct surfaces, competitive binding experiments with FL^{AgI/II} and FL^{SspB} and their fragments were analyzed by SPR using immobilized iSRCRs on CM5 sensor chip and the results of which are summarized in (FIG. 16, panels A-E). While FL^{AgI/II} was able to inhibit the binding of A₃VP₁^{AgI/II} and C₁₂₃^{AgI/II} by 46%, and 36% respectively, FL^{SspB} inhibited A₃VP₁^{SspB} and C₁₂₃^{SspB} by 54% and 23% with immobilized iSRCR₁. Similar inhibition was observed with immobilized iSRCR₁₂₃ domains where FL^{AgI/II} adherence inhibited the binding of A₃VP₁^{AgI/II} and C₁₂₃^{AgI/II} by 44% and 25%. However, FL^{SspB} had limited inhibitory effects with immobilized iSRCR₁₂₃, where A₃VP₁ and C₁₂₃ displayed 68% and 76% inhibition respectively. This points out that the surface proteins of S. mutans and S. gordonii may display different characteristics in their adherence, although they are highly homologous. In all other cases, A₃VP₁ or C₁₂₃ of AgI/II and SspB did not significantly inhibit the adherence of each other indicating that there are indeed two distinct surfaces within the SRCR domains that specifically bind AgI/II and SspBs A₃VP₁ as well as C₁₂₃ fragments.

Role of Calcium (Calcium Mediated Adherence/Stability).

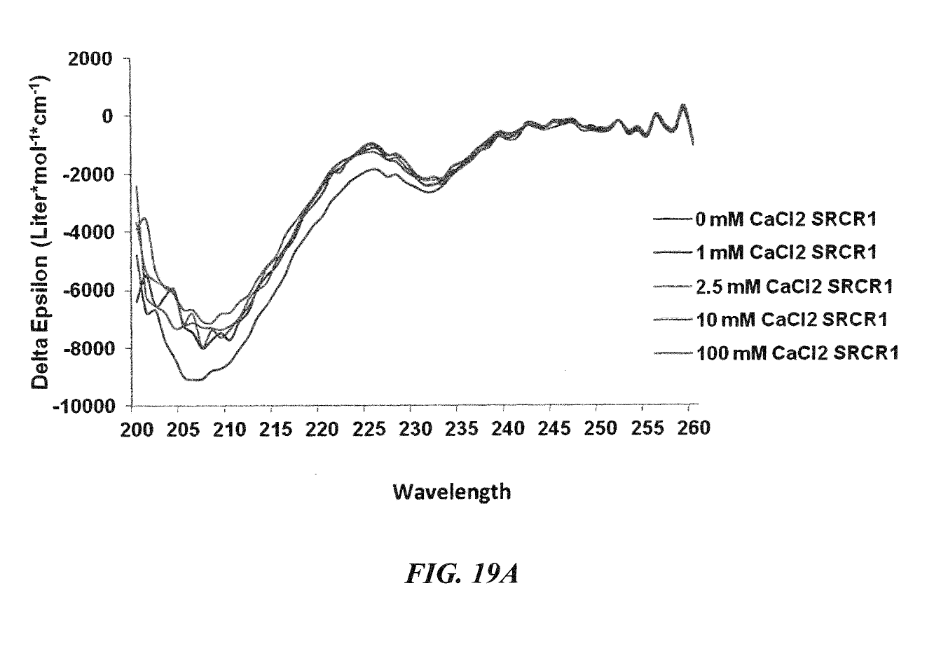

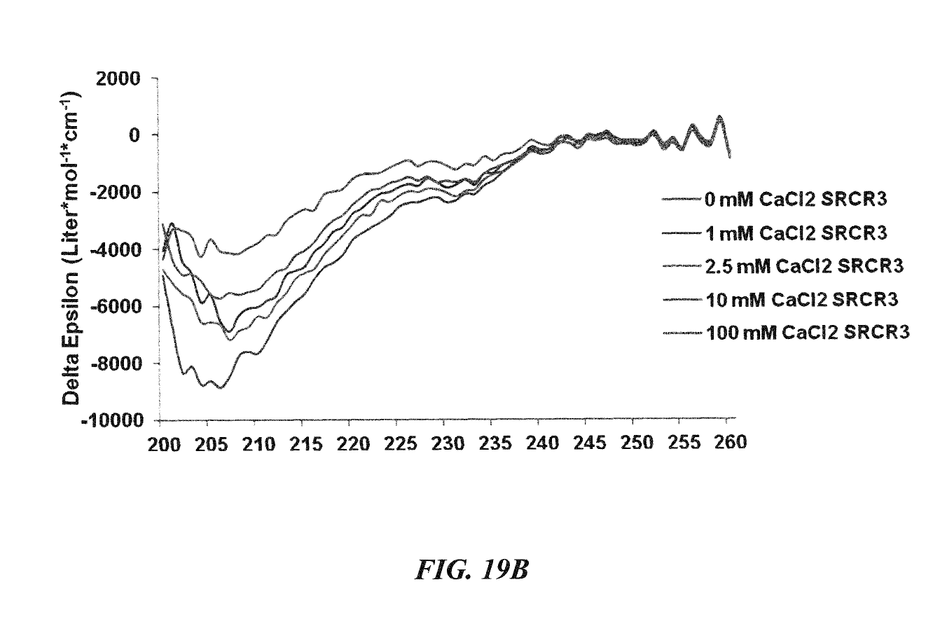

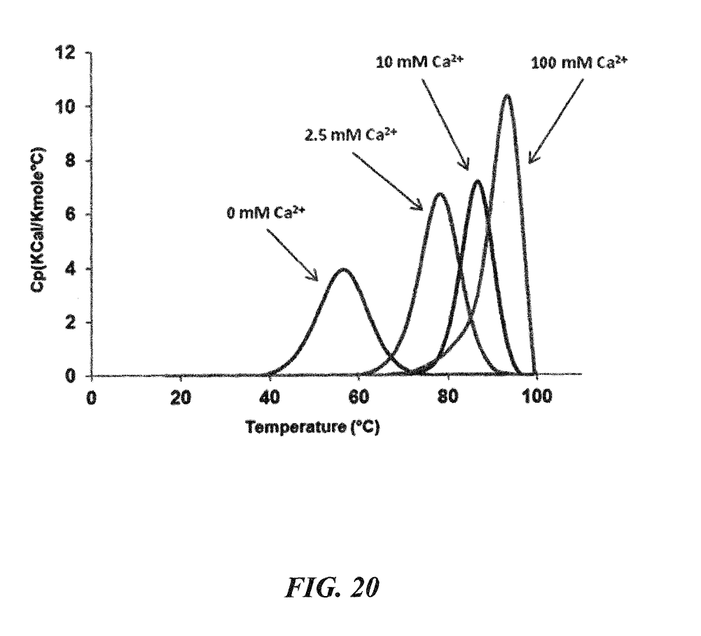

We tested to see if these SRCRs require calcium for adherence similar to that of SAG with FL and subfragments of AgI/II and SspB, and in the absence of calcium there was no adherence (FIGS. 8 and 9), and therefore we set out to determine the role of calcium ions in this adherence. In CD studies, the SRCRs clearly demonstrated a notable change in secondary structural content, particularly a reduction in alpha helices and an increase in beta sheet content in the presence of calcium (Table 4, FIG. 19, panels A and B), whereas no such changes were observed with AgI/II or SspB (data not shown). In addition, the stability (thermal unfolding) of iSRCR₁ increased in a dose dependent manner (Table 5, FIG. 20), and highly stable unfolding only at 90° C. (100 mM CaCl₂). While the thermal unfolding curves of iSRCR₁ were simple and easy to interpret, those of the iSRCR₁ was complex, with many peaks due to unfolding of multiple domains (data not shown).

TABLE 4
CD Studies on SRCRs with various calcium concentration

| | Helix (%) | | β Sheet (%) | | Turn (%) | | Random Coil (%) | |
|---|---|---|---|---|---|---|---|---|
| CaCl₂ | iSRCR₁ | iSRCR₁₂₃ | iSRCR₁ | iSRCR₁₂₃ | iSRCR₁ | iSRCR₁₂₃ | iSRCR₁ | iSRCR₁₂₃ |
| 0 mM | 15.9 | 14.8 | 28.3 | 22.9 | 20.4 | 18.2 | 35.4 | 44.2 |
| 1 mM | 6.3 | 5.9 | 34.2 | 36.4 | 24.0 | 20.3 | 35.5 | 37.3 |
| 2.5 mM | 6.6 | 6.8 | 37.4 | 34.2 | 24.2 | 20.4 | 31.9 | 38.6 |
| 10 mM | 6.8 | 5.4 | 35.9 | 34.9 | 24.3 | 19.5 | 33.0 | 40.3 |
| 100 mM | 7.2 | 4.0 | 39.3 | 36.2 | 22.2 | 18.8 | 31.3 | 41.1 |

TABLE 5
DSC Studies

| | | | Tm |
|---|---|---|---|
| Samples | ΔH | ΔHᵥ | (° C.) |
| iSRCR₁ | 5.703E⁴ ± 216 | 6.016E⁴ ± 281 | 56.5 |
| iSRCR₁ + | 7.828E⁴ ± 280 | 8.455E⁴ ± 374 | 78.1 |
| 2.5 mM CaCl₂ | | | |
| iSRCR₁ + | 6.708E⁴ ± 412 | 1.117E⁵ ± 854 | 86.4 |
| 10 mM CaCl₂ | | | |
| iSRCR₁ + | 9.736E⁴ ± 1.55E³ | 1.111E⁵ ± 2.24E³ | 92.8 |
| 100 mM CaCl₂ | | | |

While homologous structures of SRCR domains from both group A and group B have been determined [47,48], to date there are no crystal structures of the SRCR domains or Gp340. CD results summarized in Table 4 and FIG. 19, panels A and B indicate that both the iSRCR₁ and iSRCR₁₂₃ domains possess similar secondary structures compared to the solved crystal structures (PDB2JA4, PDB1BY2, PDBIP57 [47,49,50]. This indicates that the SRCR domains of Gp340 could adopt a similar SRCR fold compared to the solved crystal structures.

Effect of Carbohydrates on the Binding of AgI/II.

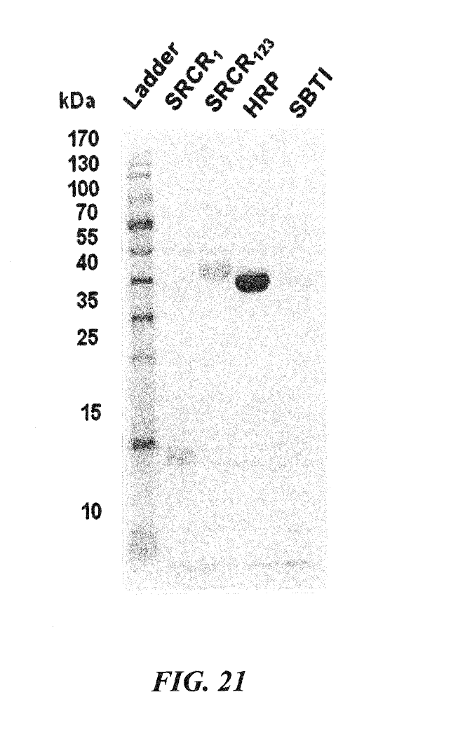

The presence of glycosylation on iSRCR₁ and iSRCR₁₂₃ was initially confirmed using glycoprotein staining (FIG. 21). While EndoH (N-glycosidase) did not have any measurable effect (data not shown), O-glycosidases had profound effects on the adherence kinetics. Deglycosylation of iSRCR₁ and iSRCR₁₂₃ by O-glycosidases did not affect the adherence characteristics of A₃VP₁ of AgI/II, but decreased the adherence of the C₁₂₃^{AgI/III} by two orders of magnitude (Table 3), indicating that carbohydrate binding could arise from the domains close to the cell surface for S. mutans AgI/II. In the case of SspB, both the apical A₃VP₁ as well as C₁₂₃ displayed reduced kinetics, and thus indicating that both these regions could possess adherence sites to carbohydrates.

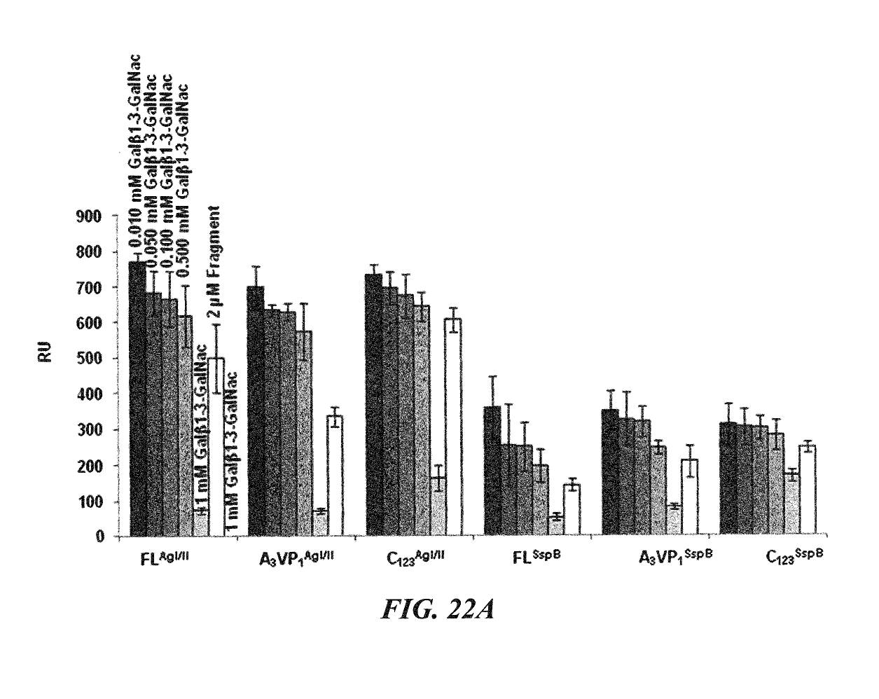

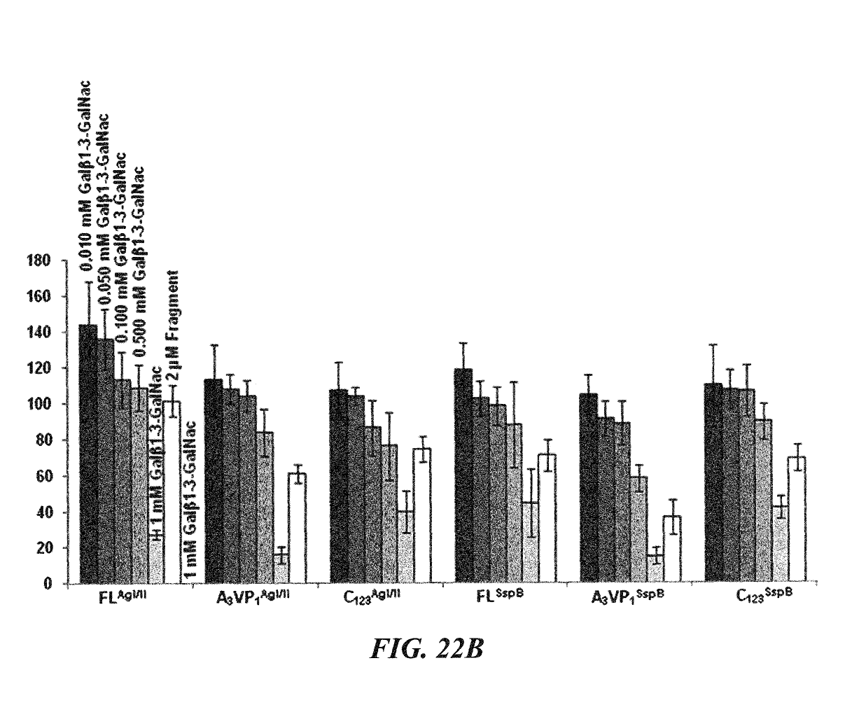

Following these above observations, glycan profile analysis of both iSRCR₁ and iSRCR₁₂₃ indicated that they are predominantly O-glycosylated with Galβ1-3-GalNac and Mannose type of carbohydrates (Table 6). Enumerating the role of Galβ1-3-GalNac and Mannose in adherence/inhibition experiments, we observed that the carbohydrate controls by themselves did not have any interactions with SRCRs, however, at lower concentrations (0.010 mM-0.500 mM) of Galβ1-3-GalNac enhanced the adhesion of all AgI/II and SspB fragments with iSRCR₁ and iSRCR₁₂₃, thus indicating an important role they play in the adhesion process. However, at 1.0 mM concentration Galβ1-3-GalNac significantly inhibited the adhesion of FL^{AgI/II}, A₃VP₁^{AgI/II} and C₁₂₃^{AgI/II} to iSRCR₁ by 85%, 79% and 73% respectively. Similarly, Galβ1-3-GalNac significantly inhibited the adhesion of FL^{SspB}, A₃VP₁^{SspB} and C₁₂₃^{SspB} to iSRCR₁ by 64%, 61% and 32% respectively. In the case of iSRCR₁₂₃ adhesion to FL^{AgI/II}, A₃VP₁^{AgI/II}, C₁₂₃^{AgI/II} was also significantly inhibited by 73%, 75% and 47%, whereas Galβ1-3-GalNac did not greatly inhibit the adhesion FL^{SspB}, A₃VP₁^{SspB} and C₁₂₃^{SspB} to iSRCR₁₂₃ (37%, 60% and 40%) (FIG. 22, panels A and B). Although, it is difficult to directly correlate these results, the apical A₃VP₁ of both AgI/II and SspB appear be the most inhibited fragment by Galβ1-3-GalNac. When testing for the role of Mannose (data now shown), it was clear that it neither inhibited nor enhanced the adhesion of FL, A₃VP₁ and C₁₂₃ of AgI/II and SspB to both iSRCR₁ and iSRCR₁₂₃.

TABLE 6
Glycan profile studies on iSRCR₁ and iSRCR₁₂₃

| iSRCR₁ | | | iSRCR₁₂₃ | | |
|---|---|---|---|---|---|
| | nmol CHO/mg | | | nmol CHO/mg | |
| Sugars | Sample | mole % | Sugars | Sample | mole % |
| | | | Fuc | 21.12 | 1.47 |
| Xylose | 23.33 | 3.45 | Xylose | 16.26 | 1.14 |
| Glucuronic acid | 85.13 | 12.58 | Glucuronic acid | 36.92 | 2.58 |
| Mannose | 156.28 | 23.10 | Mannose | 815.48 | 56.94 |
| Galactose | 108.99 | 16.11 | Galactose | 112.33 | 7.84 |
| Glucose | 1.50 | 0.22 | Glucose | 1.79 | 0.13 |
| GalNAc | 287.56 | 42.51 | GalNAc | 392.86 | 27.43 |
| GlcNAc | 13.68 | 2.02 | GlcNAc | 35.46 | 2.48 |
| SUM | 676.47 | 100.00 | SUM | 1432.22 | 100.00 |

| | | |
|---|---|---|
| mg Sample= | 1.00 | 1.00 |
| mg CHO= | 0.11 | 0.25 |
| % CHO= | 11.41 | 25.32 |

SRCRP2 (Bikker Peptide).

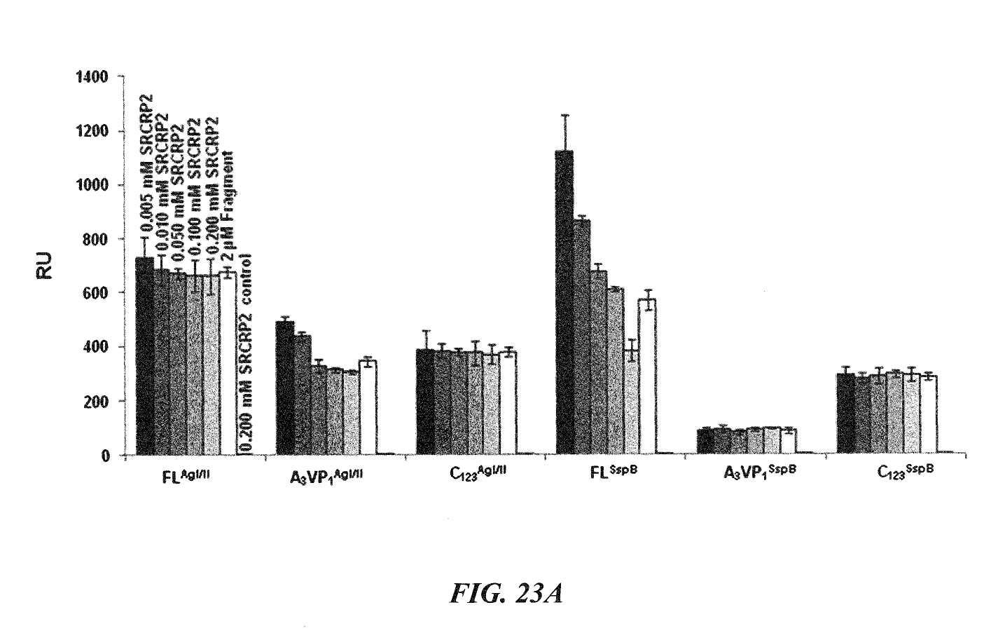

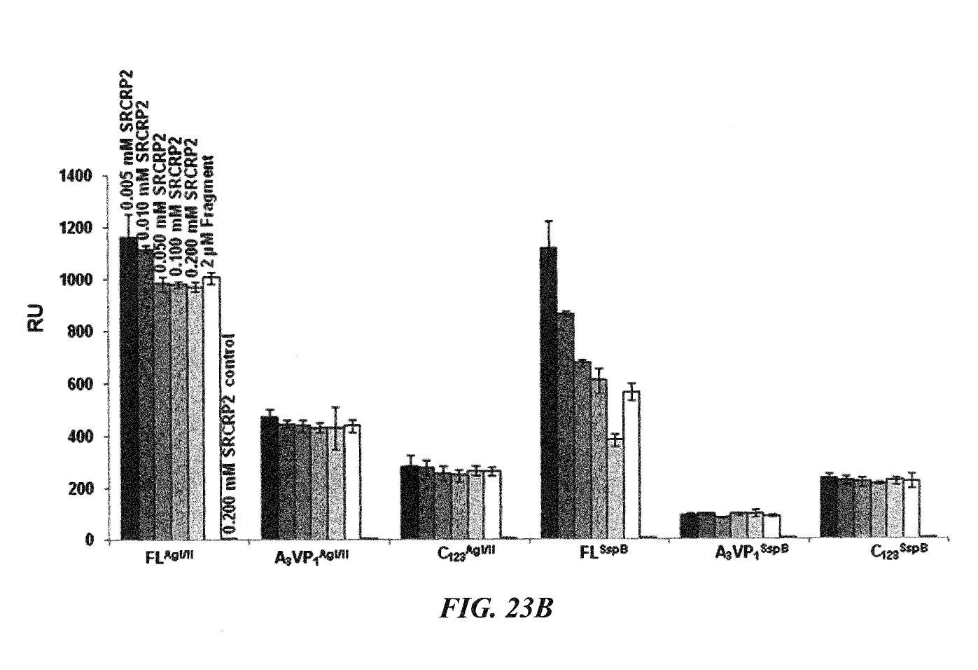

Initial ELISA assays demonstrated that the SRCRP2 peptide adheres well with AgI/II and SspB and their subfragments. When incubated at low concentration (0.005 mM) with FL^{AgI/II} and A₃VP₁^{AgI/II}, the SRCRP2 peptide improved the adherence by 8% and 13% respectively to iSRCR₁, and 8% and 16% respectively to iSRCR₁₂₃, whereas C₁₂₃^{AgI/II} had no notable changes in adherence (2.3% with iSRCR₁ and 7% with iSRCR₁₂₃). Only FL^{SspB} improved the adherence by 97% with iSRCR₁ and 91% with iSRCR₁₂₃ at lower concentration (0.005 mM), whereas A₃VP₁^{SspB} and C₁₂₃SspB did not alter the adherence characteristics to either iSRCR₁ (3% and 4%) or iSRCR₁₂₃ (3% and 5%) respectively FIG. 23, panels A and B). Also, the SRCRP2 alone (control) did not show any binding with SRCRs. These results clearly indicate that SRCRP2 peptide does not bind and inhibit the adherence of AgI/II and SspB to iSRCR₁ and iSRCR₁₂₃, indicating that the adherence site may be different from that of the aggregation sites present on AgI/II and SspB.

Self Adhesion.

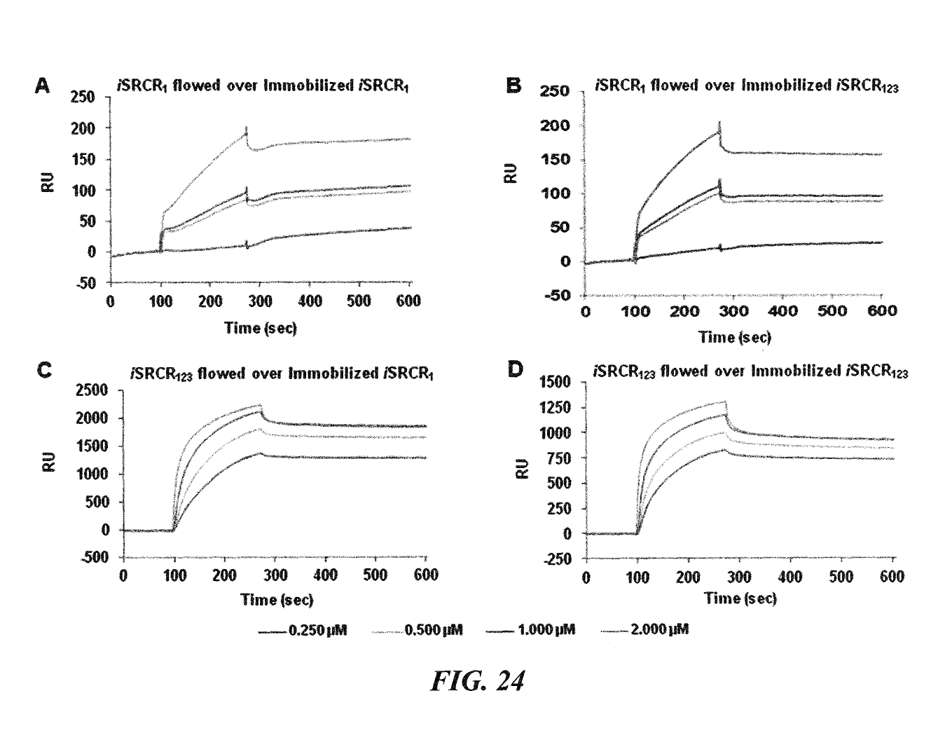

Interaction of SRCRs with each other was tested using SPR. The iSRCR₁ and iSRCR₁₂₃ strongly interacted with each other. Analytes iSRCR₁ (1.13×10⁻⁰) and iSRCR₁₂₃ (5.68×10⁻⁹) demonstrated high affinity with immobilized iSRCR₁. Similarly, analyte with iSRCR₁ (1.2×10⁻⁹) and iSRCR_{1 23} (6.72×10⁻⁹) interacted with immobilized iSRCR₁₂₃ with nanomolar affinities (FIG. 24, panels A-D). These results clearly showed that the SRCRs have self adhesion property as well.

Analytical Ultracentrifugation.

We sought to answer the question of the spatial organization of the SRCR domains, particularly whether they might be elongated similar to AgI/II through ultracentrifugation experiments. From their observed frictional ratios (iSRCR₁=1.59, iSRCR₁₂₃=1.80), resultant prolate ellipsoid ratios (iSRCR₁=7.18, iSRCR₁₂₃10.36) and calculated dimensions (iSRCR₁=12.60×1.75 nm, iSRCR₁₂₃=22.08×2.13 nm), it is clear that both iSRCR₁ and iSRCR₁₂₃ will have extended structures (Table 7). However, these may not be extended as linear rigid structures and instead may exist in a flexible non-linear conformation forming curvy tertiary structures. Models for the interaction of AgI/II and SspB with SRCR domains of Gp340 are discussed further below.

TABLE 7
Analytical Ultracentrifugation

| | | | | | | Stokes | | | Oblate | Prolate |
|---|---|---|---|---|---|---|---|---|---|---|
| | Theoretical | Fit MW | Fit rmsd | | | Radius | a/b | a/b | ellipsoid (nm × | ellipsoid (nm × |
| Construct | MW (Da) | (Da) | (OD) | S20 (S) | f/f0 | (nm) | (oblate) | (prolate) | nm) | nm) |
| iSRCR₁ | 14906 | 17031 | 0.0054 | 1.508 | 1.59 | 2.71 | 7.94 | 7.18 | 6.73 × 0.84 | 12.60 × 1.75 |
| iSRCR₁₂₃ | 42549 | 51357 | 0.0062 | 2.792 | 1.80 | 4.43 | 11.67 | 10.36 | 10.54 × 0.90 | 22.08 × 2.13 |

### C: Discussion

The ability to adhere strongly to human receptors within the oral cavity is a necessity for bacterial survival, or else they will be washed into the acidic gut [51]. Therefore bacteria that colonize the oral cavity have multiple proteins on its surface for specific adherence to human receptors [52]. As set forth herein, the oral streptococcal surface receptor AgI/II and its homologs have been examiner to develop both small molecule/peptide inhibitors as well as passive immunization [35,53,54]. The interaction AgI/II with Gp340 is considered to be the first step in adherence to tooth surface, which subsequently leads to colonization and infection, and among these the mutans streptococci are known etiological agents in dental caries [22,55]. For the past three decades this interaction has been studied using Gp340 extracted from saliva of either single or multiple donors [33,56,57].

Using recombinantly expressed SRCR domains of Gp340 by means of the Drosophila expression system, this interaction has been examined to elucidate the intricate components involved in this bacterial adhesion. The minimal AgI/II adherence region on Gp340 has been identified, and the species-specific differences in adherence among the AgI/II homologs and the role of glycolysations and metal ions have been examined.

Expression and purification of SRCRs has been reported previously [36], and herein, the specific adherence of the SRCRs using lysozyme (positive control) and thaumatin (negative control) has been shown (FIG. 14). Our results from confocal microscopic images (FIGS. 1 and 2), FACS analyses (FIG. 6) and aggregation assays (FIG. 7, panels A and B) clearly revealed that iSRCRs bind to S. mutans and S. gordonii cells, where iSRCR₁₂₃attaching more profusely compared to iSRCR₁. Through calculations based on protein adherence to chip surface (see methods) it has been discovered that higher amounts of FL^{AgI/II} and FL^{SspB} adhered to immobilized iSRCR₁₂₃ than iSRCR₁ (FIG. 15) and is in conjunction with the whole cell assays described here above. It appears from these results that more than one SRCR domain is required for efficient aggregation of bacteria. As such, longer tandem SRCR domains may more efficiently agglutinate various bacteria, and warrants further validation in future studies. Knowing that Gp340 is an innate immunity molecule, the number of tandem repeats it takes to efficiently agglutinate bacteria could have been evolutionarily determined, and it is interesting to note that in humans, Gp340 contains 14 SRCR domains, in which thirteen of them are tandem repeats, whereas in other vertebrates the number of tandem repeats are comparatively lower [24,30].

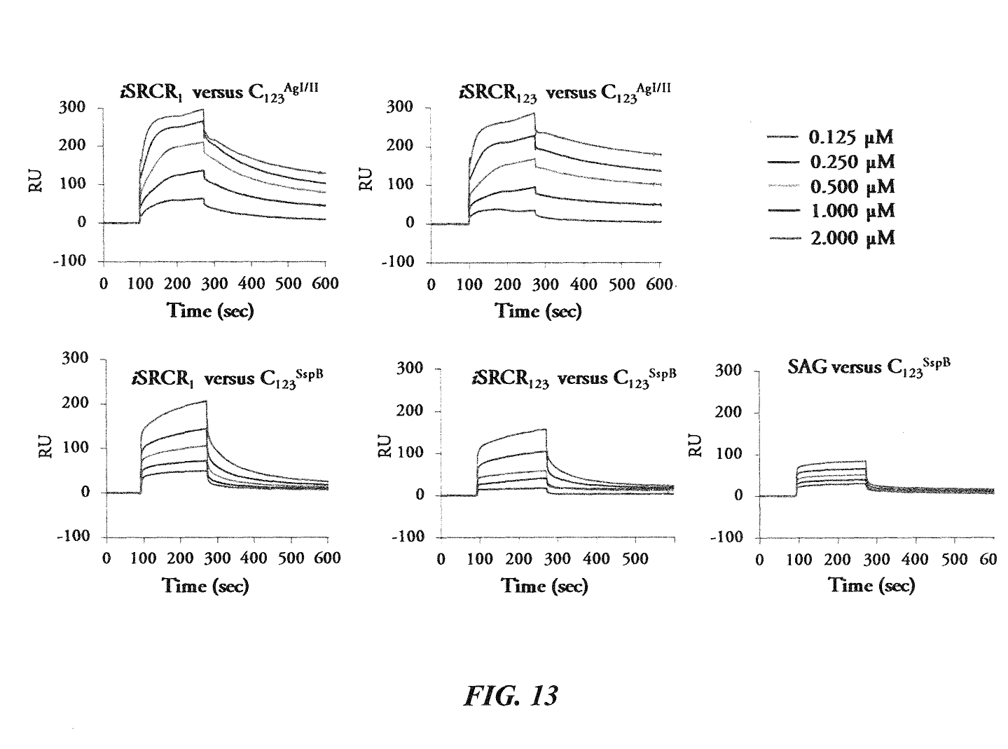

The interaction of iSRCR₁ and iSRCR₁₂₃ with AgI/II-homologs was further characterized to determine their kinetic coefficients. Initial ELISA (FIG. 9, panels A and B) assays provided clues of efficient binding of recombinant iSRCR₁ and iSRCR₁₂₃ to FL^{AgI/II} and FL^{SspB} and their individual SAG binding regions A₃VP₁ and C₁₂₃. Further characterization with SPR established the existence of nanomolar affinity interactions between the SRCRs and AgI/II-homologs (Table 3 and FIGS. 11-13. It is here that significant differences in the adherence kinetics of A₃VP₁ (apex region) of both AgI/II and SspB were discovered, whereas the adherence kinetics of the C₁₂₃ domain (present near cell the wall) to the SRCRs were not notably different. In spite of indistinguishable kinetics, it needs to be noted here that the C₁₂₃^{SspB} also displayed characteristic sensorgrams with a tendency not to remain bound to the immobilized SAG or SRCR domains (FIG. 13). The observed differences in adherence between AgI/II and the apex adherence site A₃VP₁ of SspB could be attributed to species-specific recognition and perhaps attributable to the apical V-regions of AgI/II and SspB as they are structurally distinct (37 kDa Vs 31 kDa) [58]. Overall, these results imply that the only known SAG binding protein AgI/II of pathogenic S. mutans could possibly contain a locking mechanism to maintain adherence, whereas the commensal S. gordonii, with two tandem gene repeats containing SspA and SspB, could adopt a different approach in its mode of adherence to SAG.

While it is known that A₃VP₁ and C₁₂₃ adheres to two distinct surfaces on SAG,[16], this study demonstrates that the SRCRs contains these two binding surfaces that are recognized by A₃VP₁ and C₁₂₃. These points to the fact that a single SRCR domain contains both the surfaces and is therefore the minimal adherence region for AgI/II and its homologs (FIG. 7, panels A-E). This result is highly significant as it would now render focus on the SRCR domains of Gp340 to further elucidate the multivalent adherence mechanisms of AgI/II homologs.

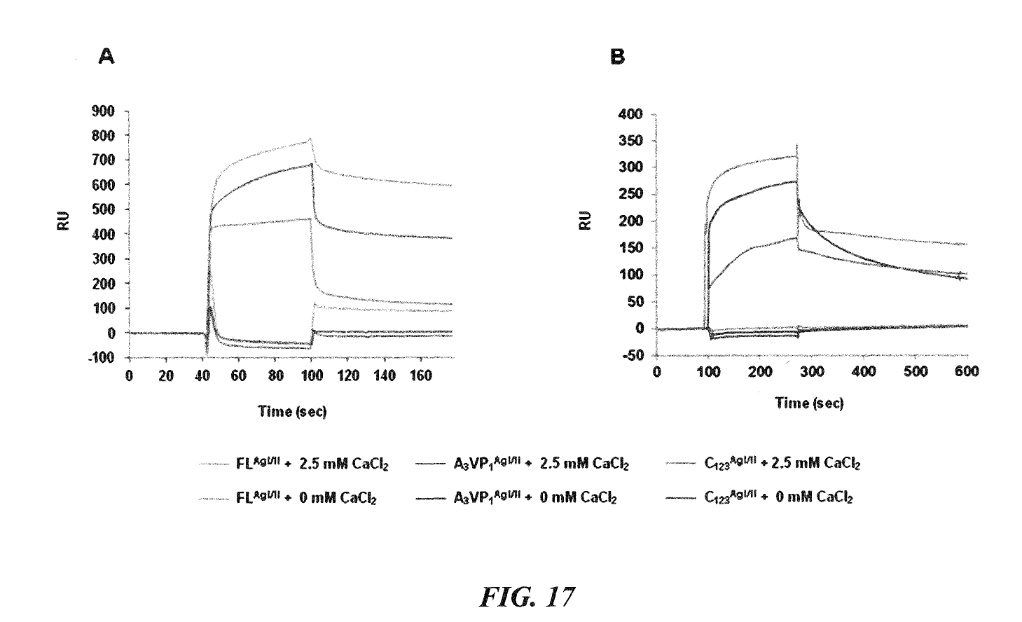

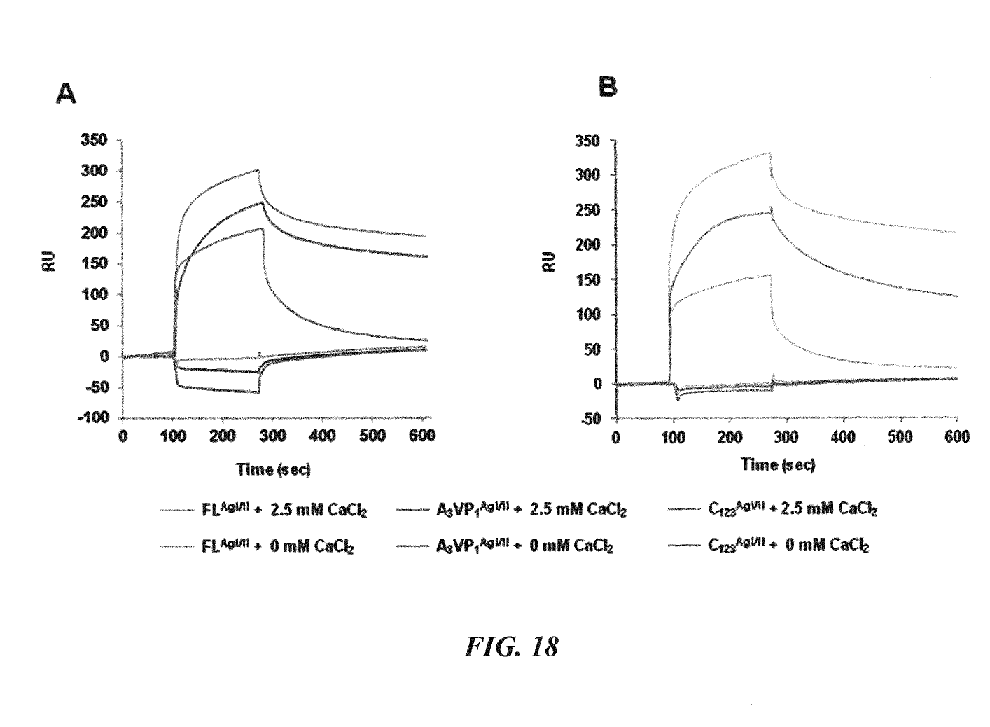

It is known that calcium plays a crucial role in the interaction between Gp340 and AgI/II homologs [59,60]. The analysis herein confirms that calcium is essential for mediating the interaction between SRCRs and AgI/II homologs (FIGS. 17 and 18). Furthermore, CD studies clearly indicate that calcium influences secondary structural changes in both iSRCR₁ and iSRCR₁₂₃ (Table 4, FIG. 19, panels A and B), and DSC analysis indicates that calcium increases the thermostability of SRCRs (Table 5, FIG. 20). These data confirm that not only the SRCRs undergo conformational change, but also get stabilized in the presence of calcium. The oral cavity is subject to environmental changes including pH and temperature. Perhaps the thermal stability that was observed may be a direct consequence of evolution, wherein these molecules have developed ability to withstand temperature changes (hot and cold food and beverages) that they face within the oral cavity. It would be interesting to see whether the SRCR domains from sea urchin possess these thermal properties which would directly link it to evolution of the SRCR domains within the human oral cavity.

Gp340 is decorated with glycosylations [24,61], and were previously shown to play an important role in the adherence of AgI/II and its homologs [62]. While glycostaining of recombinant SRCRs indicated the presence of glycans (FIG. 21), we further expounded their composition using glycan profile analysis (Table 6), which showed predominant O-glycosylation. Deglycosylation with O-glycanase reduced the adherence of AgI/II and SspB by 10 fold (Table 3), whereas the deglycosylation with EndoH (N-glycanase) had no significant effect (data not shown) thus indicating a role for the glycosylations. While these results implicate O-glycosylations to be the major player, it is not possible to currently rule out the effect of N-glycosylations in adhesion. In a series of further experiments, where AgI/II and SspB and their sub-fragments were incubated with various concentrations of Galβ1-3-GalNac, it appears that there seems to be a significant additive effect (more of AgI/II adhered) at lower concentrations of the glycosylations, indicating a role in adhesion, as well as inhibition at higher concentrations with SRCRs, possibly saturating the adherence sites (FIG. 22, panels A and B). These above exemplify that glycosylations play a role, albeit peripheral compared to the calcium ions, in adherence.

The SRCRP2 peptide has been shown to adhere and aggregate bacteria [20,34]. However, the aggregation by the SRCRP2 peptide (FIG. 6, panels A and B) was very limited, even at high concentration (FIG. 3), compared to that of iSRCR₁ and iSRCR₁₂₃, indicating that the folded protein may possess additional sites that result in efficient aggregation. While fluorescence based assays indicated adherence of the SRCRP2 peptide to AgI/II and SspB (FIG. 9), incubation of SRCRP2 peptides with the apex (A₃VP₁) and proximal cell surface (C₁₂₃) SAG adherence domains did not significantly inhibit the adherence, but rather surprisingly increased the adhesiveness of only FL^{SspB}, indicating the presence of a non-specific aggregation property (FIG. 22, panels A and B). These results now demonstrate that the peptide while possessing an aggregating property, is very limited in its ability compared to the full length folded protein domains, and more importantly it neither adheres to nor blocks the GP340 binding motif/site on either AgI/II or SspB.

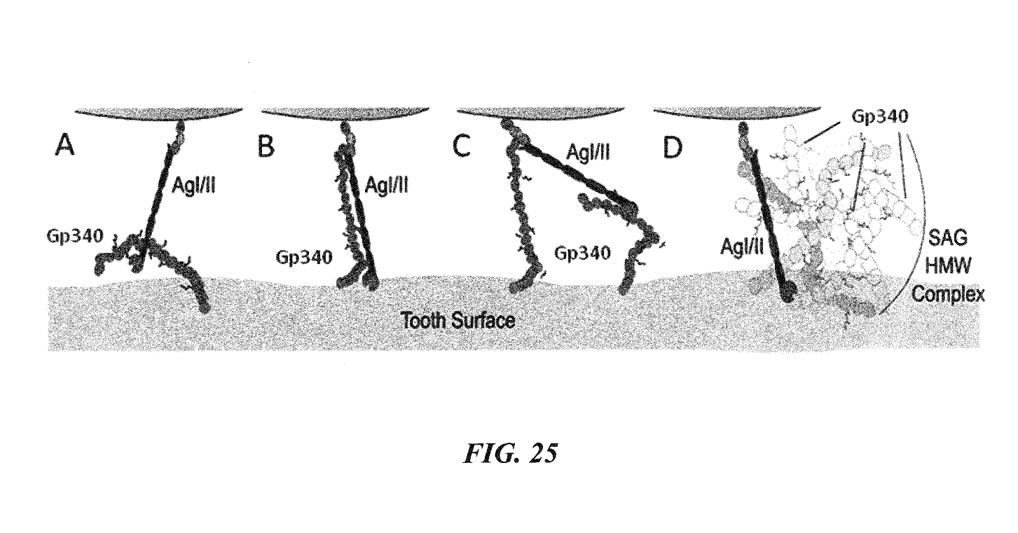

It is known that Gp340 exists as a higher order complex, and these aggregates could be as large as 5000 kDa [22,63]. The aggregation property of Gp340 has been attributed to the Zona Pellucida (ZP) domain, as in other mammalian proteins, the ZP domain was shown to be involved in self-aggregation [64]. In this context, we tested for the ability of the SRCR domains for self-interaction, and surprisingly found that they associate with nano-molar affinities, and thus indicating that this association as highly specific, as non-specific interactions traditionally appears to fall within the micro-molar range (FIG. 24, panels A-D). These results now for the first time implicate the SRCR domains in self-aggregation, and opens up several possible models for bacterial aggregation, wherein one potential model could simulate the bacterial proteins to be sandwiched between two SRCRs (Gp340s) (FIG. 25). It is here that the tertiary architecture of tandem SRCR domains were examined, and identified through ultracentrifugation studies that the SRCR domains may not strictly form a linear elongated structure (Table 7) but could form a curvy centipede-like extended structure, similar to that observed in electron microscopy images of Gp340 [31].

While that which is exemplified herein is generally focused on AgI/II and its homologs, it has been shown that the SRCR domains of Gp340 play a pivotal role in mediating HIV adhesion/clearance through Gp120 within the oral cavity [65,66]. While Gp340 acted as a clearance mechanism in the oral cavity, the case was very different on the vaginally derived Gp340, which is immobilized on the cell surface, where this was shown to mediate trancytosis from apical to basolateral surface in both genital tract epithelial cells in culture and with endocervical tissue [67]. Similarly, in our SPR experiments, immobilized SRCRs adhere tightly to AgI/II homologs, while in fluid phase SRCRs aggregate S. mutans and S. gordonii, a double faceted property, where on the one hand acts as a portal of entry for microbes while immobilized, on the other as a clearance mechanism within the oral cavity in fluid state. This property indicates that SRCRs may adopt different secondary structural conformations in fluid and immobilized states and this conformation could be induced by calcium ions.

That which is exemplified herein indicates that the minimal adherence region is restricted to a single SRCR domain, which carries the two distinct surfaces that adhere to A₃VP₁ as well as C₁₂₃ of both AgI/II and SspB with increasing number of SRCR domains for better adherence and aggregation of bacteria. Calcium mediated structural changes are essential for the adherence of AgI/II and SspB, and the SRCR domains become more stable at higher concentrations of calcium. Biophysical characterization indicates that the SRCR domains may adopt a curvy centipede like structure. That which is exemplified herein also establish that glycosylations do play a role in the adherence to AgI/II and SspB. While there are similarities in the binding of AgI/II and SspB, there are certainly distinct differences pointing toward species specificity in their adherence. Overall, that which is exemplified herein indicate that focusing on the SRCR domains and the interactions at a molecular level between AgI/II homologs and SRCR can assist in identifying interventional therapeutics in the form of small molecule inhibitors, or development of passive immunization therapies that can impede oral streptococcal adherence to tooth surfaces and alleviate the global burden of dental caries.

### EXAMPLE 1 REFERENCES

1. Brooks W, Demuth D R, Gil S, Lamont R J (1997) Identification of a Streptococcus gordonii SspB domain that mediates adhesion to Porphyromonas gingivalis. Infect Immun 65: 3753-3758.
2. Chung W O, Demuth D R, Lamont R J (2000) Identification of a Porphyromonas gingivalis receptor for the Streptococcus gordonii SspB protein. Infect Immun 68: 6758-6762.
3. Demuth D R, Duan Y, Brooks W, Holmes A R, McNab R, et al. (1996) Tandem genes encode cell-surface polypeptides SspA and SspB which mediate adhesion of the oral bacterium Streptococcus gordonii to human and bacterial receptors. Mol Microbiol 20: 403-413.
4. Demuth D R, Duan Y, Jenkinson H F, McNab R, Gil S, et al. (1997) Interruption of the Streptococcus gordonii M5 sspA/sspB intergenic region by an insertion sequence related to IS1167 of Streptococcus pneumoniae. Microbiology 143 (Pt 6): 2047-2055.
5. Egland P G, Du L D, Kolenbrander P E (2001) Identification of independent Streptococcus gordonii SspA and SspB functions in coaggregation with Actinomyces naeslundii. Infect Immun 69: 7512-7516.
6. El-Sabaeny A, Demuth D R, Lamont R J (2001) Regulation of Streptococcus gordonii sspB by the sspA gene product. Infect Immun 69: 6520-6522.
7. Lamont R J, El-Sabaeny A, Park Y, Cook G S, Costerton J W, et al. (2002) Role of the Streptococcus gordonii SspB protein in the development of Porphyromonas gingivalis biofilms on streptococcal substrates. Microbiology 148: 1627-1636.
8. Lehner T, Russell M W, Caldwell J (1980) Immunisation with a purified protein from Streptococcus mutans against dental caries in rhesus monkeys. Lancet 1: 995-996.
9. Russell M W, Childers N K, Michalek S M, Smith D J, Taubman M A (2004) A Caries Vaccine? The state of the science of immunization against dental caries. Caries Res 38: 230-235.
10. Robinette R A, Oli M W, McArthur W P, Brady L J (2011) A therapeutic anti-Streptococcus mutans monoclonal antibody used in human passive protection trials influences the adaptive immune response. Vaccine 29: 6292-6300.
11. Brady L J, Maddocks S E, Larson M R, Forsgren N, Persson K, et al. (2010) The changing faces of Streptococcus antigen I/II polypeptide family adhesins. Mol Microbiol 77: 276-286.
12. Nobbs A H, Lamont R J, Jenkinson H F (2009) Streptococcus adherence and colonization. Microbiol Mol Biol Rev 73: 407-450, Table of Contents.
13. Ericson T, Rundegren J (1983) Characterization of a salivary agglutinin reacting with a serotype c strain of Streptococcus mutans. Eur J Biochem 133: 255-261.
14. Mollenhauer J, Wiemann S, Scheurlen W, Korn B, Hayashi Y, et al. (1997) DMBT1, a new member of the SRCR superfamily, on chromosome 10q25.3-26.1 is deleted in malignant brain tumours. Nat Genet 17: 32-39.
15. Larson M R, Rajashankar K R, Patel M H, Robinette R A, Crowley P J, et al. (2010) Elongated fibrillar structure of a streptococcal adhesin assembled by the high-affinity association of alpha- and PPII-helices. Proc Natl Acad Sci USA 107: 5983-5988.
16. Larson M R, Rajashankar K R, Crowley P J, Kelly C, Mitchell T J, et al. (2011) Crystal structure of the C-terminal region of Streptococcus mutans antigen I/II and characterization of salivary agglutinin adherence domains. J Biol Chem 286: 21657-21666.
17. Deivanayagam C C, Wann E R, Chen W, Carson M, Rajashankar K R, et al. (2002) A novel variant of the immunoglobulin fold in surface adhesins of Staphylococcus aureus: crystal structure of the fibrinogen-binding MSCRAMM, clumping factor A. Embo J 21: 6660-6672.
18. Leito J T, Ligtenberg A J, van Houdt M, van den Berg T K, Wouters D (2011) The bacteria binding glycoprotein salivary agglutinin (SAG/gp340) activates complement via the lectin pathway. Mol Immunol 49: 185-190.
19. Wu Z, Golub E, Abrams W R, Malamud D (2004) gp340 (SAG) binds to the V3 sequence of gp120 important for chemokine receptor interaction. AIDS Res Hum Retroviruses 20: 600-607.
20. Bikker F J, Ligtenberg A J, Nazmi K, Veerman E C, van't Hof W, et al. (2002) Identification of the bacteria-binding peptide domain on salivary agglutinin (gp-340/DMBT1), a member of the scavenger receptor cysteine-rich superfamily. J Biol Chem 277: 32109-32115.
21. Prakobphol A, Xu F, Hoang V M, Larsson T, Bergstrom J, et al. (2000) Salivary agglutinin, which binds Streptococcus mutans and Helicobacter pylori, is the lung scavenger receptor cysteine-rich protein gp-340. J Biol Chem 275: 39860-39866.
22. Oho T, Yu H, Yamashita Y, Koga T (1998) Binding of salivary glycoprotein-secretory immunoglobulin A complex to the surface protein antigen of Streptococcus mutans. Infect Immun 66: 115-121.
23. Demuth D R, Lammey M S, Huck M, Lally E T, Malamud D (1990) Comparison of Streptococcus mutans and Streptococcus sanguis receptors for human salivary agglutinin. Microb Pathog 9: 199-211.
24. Ligtenberg A J, Karlsson N G, Veerman E C Deleted in Malignant Brain Tumors-1 Protein (DMBT1): A Pattern Recognition Receptor with Multiple Binding Sites. Int J Mol Sci 11: 5212-5233.
25. Claudianos C, Dessens J T, Trueman H E, Arai M, Mendoza J, et al. (2002) A malaria scavenger receptor-like protein essential for parasite development. Mol Microbiol 45: 1473-1484.
26. Abrahamsen M S, Templeton T J, Enomoto S, Abrahante J E, Zhu G, et al. (2004) Complete genome sequence of the apicomplexan, Cryptosporidium parvum. Science 304: 441-445.
27. Hohenester E, Sasaki T, Timpl R (1999) Crystal structure of a scavenger receptor cysteine-rich domain sheds light on an ancient superfamily. Nat Struct Biol 6: 228-232.
28. Stoddard E, Cannon G, Ni H, Kariko K, Capodici J, et al. (2007) gp340 expressed on human genital epithelia binds HIV-1 envelope protein and facilitates viral transmission. J Immunol 179: 3126-3132.
29. Cummins J E, Christensen L, Lennox J L, Bush T J, Wu Z, et al. (2006) Mucosal innate immune factors in the female genital tract are associated with vaginal HIV-1 shedding independent of plasma viral load. AIDS Res Hum Retroviruses 22: 788-795.
30. Ligtenberg A J, Veerman E C, Nieuw Amerongen A V, Mollenhauer J (2007) Salivary agglutinin/glycoprotein-340/DMBT1: a single molecule with variable composition and with different functions in infection, inflammation and cancer. Biol Chem 388: 1275-1289.
31. Madsen J, Mollenhauer J, Holmskov U (2010) Review: Gp-340/DMBT1 in mucosal innate immunity. Innate Immun 16: 160-167.
32. Malamud D, Abrams W R, Barber C A, Weissman D, Rehtanz M, et al. Antiviral activities in human saliva. Adv Dent Res 23: 34-37.
33. Loimaranta V, Jakubovics N S, Hytonen J, Finne J, Jenkinson H F, et al. (2005) Fluid- or surface-phase human salivary scavenger protein gp340 exposes different bacterial recognition properties. Infect Immun 73: 2245-2252.
34. Bikker F J, Ligtenberg A J, End C, Renner M, Blaich S, et al. (2004) Bacteria binding by DMBT1/SAG/gp-340 is confined to the VEVLXXXXW motif in its scavenger receptor cysteine-rich domains. J Biol Chem 279: 47699-47703.
35. Jakubovics N S, Stromberg N, van Dolleweerd C J, Kelly C G, Jenkinson H F (2005) Differential binding specificities of oral streptococcal antigen I/II family adhesins for human or bacterial ligands. Mol Microbiol 55: 1591-1605.
36. Purushotham S, Deivanayagam C (2013) Cloning, expression and purification of the SRCR domains of glycoprotein 340. Protein Expr Purif 90: 67-73.
37. Brady L J, Piacentini D A, Crowley P J, Oyston P C, Bleiweis A S (1992) Differentiation of salivary agglutinin-mediated adherence and aggregation of mutans streptococci by use of monoclonal antibodies against the major surface adhesin P1. Infect Immun 60: 1008-1017.
38. Oli M W, McArthur W P, Brady L J (2006) A whole cell BIAcore assay to evaluate P1-mediated adherence of Streptococcus mutans to human salivary agglutinin and inhibition by specific antibodies. J Microbiol Methods 65: 503-511.
39. Schuck P (1997) Use of surface plasmon resonance to probe the equilibrium and dynamic aspects of interactions between biological macromolecules. Annual Review of Biophysics and Biomolecular Structure 26: 541-566.
40. Sreerama N, Woody R W (2000) Estimation of protein secondary structure from circular dichroism spectra: comparison of CONTIN, SELCON, and CDSSTR methods with an expanded reference set. Anal Biochem 287: 252-260.
41. Yang Z R W, Tendian S W, Carson W M, Brouillette W J, Delucas L J, et al. (2004) Dimethyl sulfoxide at 2.5% (v/v) alters the structural cooperativity and unfolding mechanism of dimeric bacterial NAD(+) synthetase. Protein Science 13: 830-841.
42. Hobb R I, Tzeng Y L, Choudhury B P, Carlson R W, Stephens D S (2010) Requirement of NMB0065 for connecting assembly and export of sialic acid capsular polysaccharides in Neisseria meningitidis. Microbes Infect 12: 476-487.
43. Lebowitz J, Lewis M S, Schuck P (2002) Modern analytical ultracentrifugation in protein science: a tutorial review. Protein science: a publication of the Protein Society 11: 2067-2079.
44. Schuck P (2000) Size-distribution analysis of macromolecules by sedimentation velocity ultracentrifugation and lamm equation modeling. Biophys J 78: 1606-1619.
45. Jenkinson H F, Demuth D R (1997) Structure, function and immunogenicity of streptococcal antigen I/II polypeptides. Mol Microbiol 23: 183-190.
46. Ahn S J, Ahn S J, Wen Z T, Brady L J, Burne R A (2008) Characteristics of biofilm formation by Streptococcus mutans in the presence of saliva. Infect Immun 76: 4259-4268.
47. Rodamilans B, Munoz I G, Bragado-Nilsson E, Sarrias M R, Padilla O, et al. (2007) Crystal structure of the third extracellular domain of CD5 reveals the fold of a group B scavenger cysteine-rich receptor domain. J Biol Chem 282: 12669-12677.
48. Resnick D, Pearson A, Krieger M (1994) The SRCR superfamily: a family reminiscent of the Ig superfamily. Trends Biochem Sci 19: 5-8.
49. Hohenester E, Sasaki T, Timpl R (1999) Crystal structure of a scavenger receptor cysteine-rich domain sheds light on an ancient superfamily. Nature structural biology 6: 228-232.
50. Somoza J R, Ho J D, Luong C, Ghate M, Sprengeler P A, et al. (2003) The structure of the extracellular region of human hepsin reveals a serine protease domain and a novel scavenger receptor cysteine-rich (SRCR) domain. Structure 11: 1123-1131.
51. Moore W E, Moore L V (1994) The bacteria of periodontal diseases. Periodontol 2000 5: 66-77.
52. Whittaker C J, Klier C M, Kolenbrander P E (1996) Mechanisms of adhesion by oral bacteria. Annu Rev Microbiol 50: 513-552.
53. Pearce C, Bowden G H, Evans M, Fitzsimmons S P, Johnson J, et al. (1995) Identification of pioneer viridans streptococci in the oral cavity of human neonates. J Med Microbiol 42: 67-72.
54. Holmes A R, Gilbert C, Wells J M, Jenkinson H F (1998) Binding properties of Streptococcus gordonii SspA and SspB (antigen I/II family) polypeptides expressed on the cell surface of Lactococcus lactis MG1363. Infect Immun 66: 4633-4639.
55. Lamont R J, Demuth D R, Davis C A, Malamud D, Rosan B (1991) Salivary-agglutinin-mediated adherence of Streptococcus mutans to early plaque bacteria. Infect Immun 59: 3446-3450.
56. Esberg A, Lofgren-Burstrom A, Ohman U, Stromberg N (2012) Host and bacterial phenotype variation in adhesion of Streptococcus mutans to matched human hosts. Infect Immun 80: 3869-3879.
57. Ligtenberg T J, Bikker F J, Groenink J, Tornoe I, Leth-Larsen R, et al. (2001) Human salivary agglutinin binds to lung surfactant protein-D and is identical with scavenger receptor protein gp-340. Biochem J 359: 243-248.
58. Forsgren N, Lamont R J, Persson K (2009) Crystal structure of the variable domain of the Streptococcus gordonii surface protein SspB. Protein Sci 18: 1896-1905.
59. Rundegren J (1986) Calcium-dependent salivary agglutinin with reactivity to various oral bacterial species. Infect Immun 53: 173-178.
60. Duan Y, Fisher E, Malamud D, Golub E, Demuth D R (1994) Calcium-binding properties of SSP-5, the Streptococcus gordonii M5 receptor for salivary agglutinin. Infect Immun 62: 5220-5226.
61. Holmskov U, Mollenhauer J, Madsen J, Vitved L, Gronlund J, et al. (1999) Cloning of gp-340, a putative opsonin receptor for lung surfactant protein D. Proc Natl Acad Sci USA 96: 10794-10799.
62. Oho T, Bikker F J, Nieuw Amerongen A V, Groenink J (2004) A peptide domain of bovine milk lactoferrin inhibits the interaction between streptococcal surface protein antigen and a salivary agglutinin peptide domain. Infect Immun 72: 6181-6184.
63. Young A, Rykke M, Smistad G, Rolla G (1997) On the role of human salivary micelle-like globules in bacterial agglutination. Eur J Oral Sci 105: 485-494.
64. Jovine L, Darie C C, Litscher E S, Wassarman P M (2005) Zona pellucida domain proteins. Annu Rev Biochem 74: 83-114.
65. Wu Z, Lee S, Abrams W, Weissman D, Malamud D (2006) The N-terminal SRCR-SID domain of gp-340 interacts with HIV type 1 gp120 sequences and inhibits viral infection. AIDS Res Hum Retroviruses 22: 508-515.
66. Wu Z, Golub E, Abrams W R, Malamud D (2004) gp340 (SAG) binds to the V3 sequence of gp120 important for chemokine receptor interaction. AIDS research and human retroviruses 20: 600-607.
67. Stoddard E, Ni H, Cannon G, Zhou C, Kallenbach N, et al. (2009) gp340 promotes transcytosis of human immunodeficiency virus type 1 in genital tract-derived cell lines and primary endocervical tissue. Journal of virology 83: 8596-8603.

### Example 2

### Inhibition of SAG Adherence to Other Bacterial Surface Proteins by Galβ1-3GalNac (Core-1)

Inhibition of SAG adherence to AgI/II, SspB, Pas, along with glucan binding protein C (GbpC) and collagen binding protein (rcnM) by Galβ1-3GalNac (core-1) was determined as is described in Example 1 and as described below.

Method:

Recombinant full length (FL) constructs of surface proteins (homologs of AgI/II) AgI/II, SspB, Pas, along with GbpC (Glucan binding protein C) and rcnM (collagen binding protein) were incubated with various concentrations (0.010 mM to 2 mM) of Galβ1-3GalNac at room temperature and their binding interaction with immobilized salivary agglutinin on a CM5 sensor chip was determined. In control experiments, 2 mM Galβ1-3GalNac did not show direct adherence to SAG.

Results:

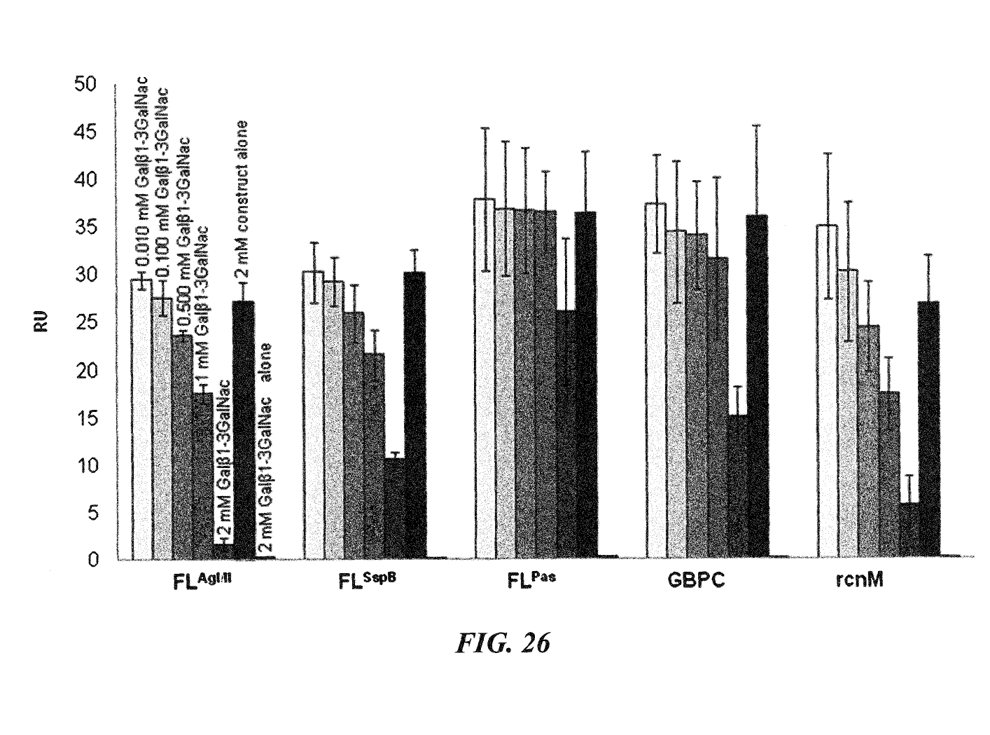

Results of SAG inhibition on other bacterial surface proteins are depicted in FIG. 1. At a concentration of 2 mM, Galβ1-3GalNac inhibited SAG adherence of AgI/II by 94%, SspB by 65%, GBPC by 72%, rcnM by 69%, another AgI/II homolog, Pas, exhibited 28% inhibition. These results in the inhibition SAG adherence by Galβ1-3GalNac shown in FIG. 26 clearly point towards the use of Galβ1-3GalNac as a broad range inhibitor of SAG adherence that can target more than one surface protein. In addition, these results are: indicative of how each surface protein interacts with SAG through carbohydrates; indicative that Galβ1-3GalNac can effectively inhibit attachment of pathogenic oral streptococci to SAG; and indicative that Galβ1-3GalNac can serve the worldwide populace with dental caries.

### Example 3

### Binding of SRCR Peptide to AgI/II, SspB and GbpC

Binding of the SRCR peptide ETNDANVVARQL (SEQ ID NO: 10) to immobilized bacterial surface proteins was determined using procedures as described in Example 1.

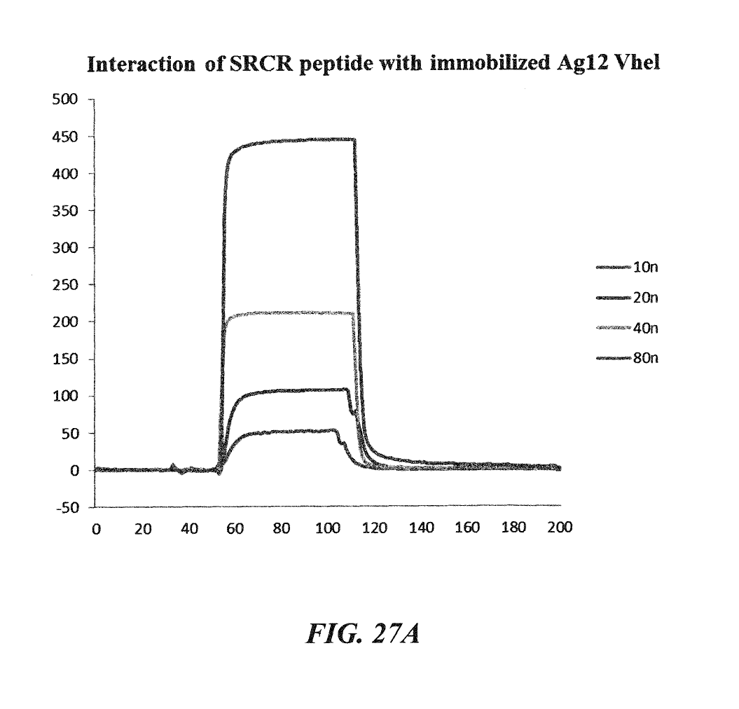

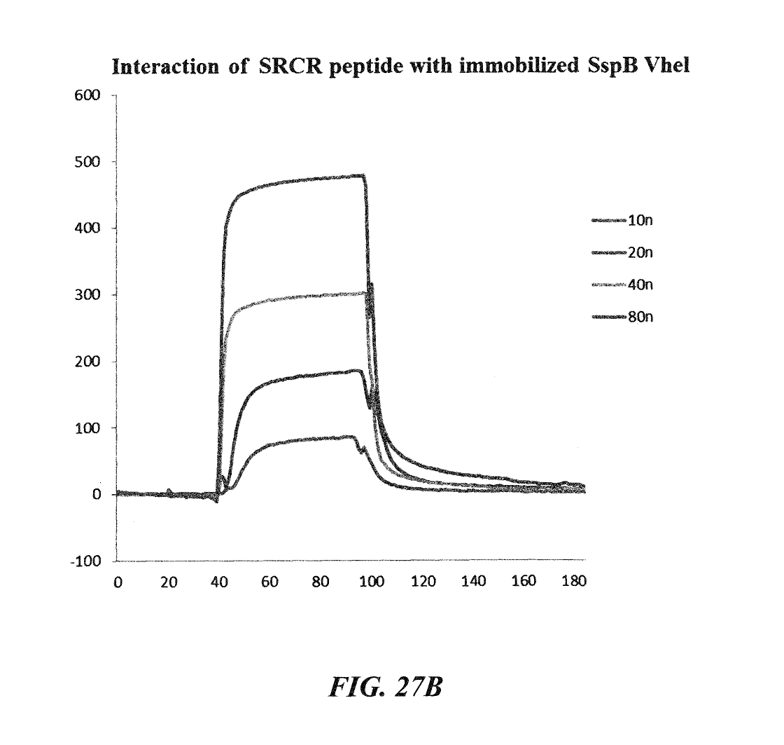

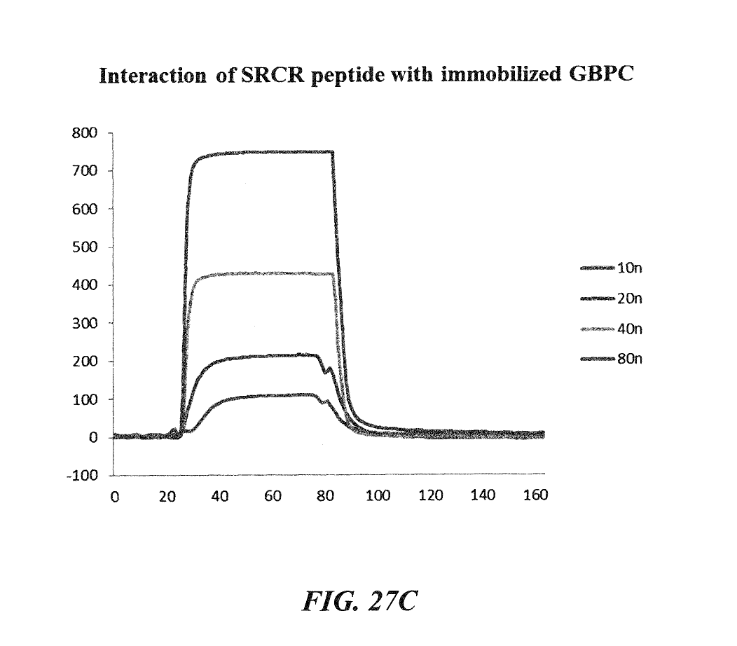

The interaction of the SRCR peptide ETNDANVVARQL (SEQ ID NO:10) with immobilized bacterial surface proteins AgI/II, SspB and GbpC as examined by surface plasmon resonance studies are shown in FIGS. 27A, 27B and 27C respectively, and binding affinities determined from these studies are listed in Table 8.

TABLE 8
Surface plasmon resonance studies with SRCR peptide

| | kₐ (1/ms) | k_{d}(1/s) | Rmax (Ru) | K_{A} (1/M) | K_{D} (M) | Chi2 |
|---|---|---|---|---|---|---|
| AgI/II VheI (S. mutans) | 1.08 × 10⁷ | 0.696 | 43.3 | 1.56 × 10⁸ | 6.42 × 10⁻⁹ | 8.72 |
| SspB VheI (S. gordonii) | 4.44 × 10⁶ | 0.0312 | 110 | 1.42 × 10⁸ | 7.02 × 10⁻⁹ | 6.99 |
| GbpC (S. mutans) | 1.47 × 10⁷ | 0.0573 | 232 | 2.57 × 10⁸ | 3.89 × 10⁻⁹ | 25.1 |

As shown by the dissociation constants K_{D} listed in Table 8, the SRCR peptide ETNDANVVARQL (SEQ ID NO:10) binds with nanomolar affinity (3.89-7.02×10⁻⁹ M) to bacterial surface proteins AgI/II, SspB and GbpC.

These results indicate that the peptide ETNDANVVARQL (SEQ ID NO: 10) may be a peptide inhibitor of the interaction between AgI/II, and its homologs, at SAG.

The foregoing is illustrative of the present invention, and is not to be construed as limiting thereof. The invention is defined by the following claims, with equivalents of the claims to be included therein.

1. A composition comprising an inhibitor of the interaction of AgI/II, or a homolog thereof, with salivary agglutinin (SAG).

2. The composition of claim 1, wherein the homolog of AgI/II is selected from the group consisting of SspB, Pas, GBPC and rcnM, or a combination of any thereof.

3. The composition of claim 1, wherein the inhibitor interacts with glycoprotein 340 (Gp340) found within an SAG glycoprotein complex.

4. The composition of claim 1, wherein the inhibitor interacts with a scavenger receptor cysteine rich (SRCR) domain present on Gp340.

5. The composition of claim 1, wherein the inhibitor interacts with AgI/II, or homolog thereof.

6. The composition of claim 1, wherein the inhibitor comprises a glycan.

7. The composition of claim 6, wherein the glycan is Galβ1-3-GalNac.

8. The composition of claim 1, wherein the inhibitor comprises a peptide.

9. The composition of claim 8, wherein the peptide interacts with AgI/II, or homolog thereof.

10. The composition of claim 8, wherein the peptide is ETNDANVVARQL (SEQ ID NO:10).

11. A formulation comprising the composition of claim 1.

12. The formulation of claim 11, wherein the formulation is in a form suitable for oral or buccal (sublingual) administration.

13. The formulation of claim 11, wherein the formulation is in a form of a tooth paste, oral rinse, gel, an additive to a digestible product or a strip comprising the formulation to be applied to the teeth of a subject.

14. A method of preventing, inhibiting or treating the formation of dental caries in a subject comprising the administration of a composition or formulation comprising an inhibitor of the interaction of AgI/II, or a homolog thereof, with SAG.

15. The method of claim 14, wherein the homolog of AgI/II is selected from the group consisting of SspB, Pas, GBPC and rcnM, or a combination of any thereof.

16. The method of claim 14, wherein the inhibitor comprises a glycan.

17. The method of any one of claim 16, wherein the glycan is Galβ1-3-GalNac.

18. The method of claim 14, wherein the inhibitor comprises a peptide.

19. The method of claim 18, wherein the peptide is ETNDANVVARQL (SEQ ID NO:10).

20. The method of claim 14, wherein the composition or formulation is administered in a form selected from the group consisting of a tooth paste, oral rinse, gel, an additive to a digestible product and a strip comprising the formulation to be applied to the teeth of a subject.

21. The method of claim 14, wherein the subject is a human subject.

22. A method of preventing or inhibiting the formation of denture plaques comprising the administration of or treatment with a composition or formulation comprising an inhibitor of the interaction of AgI/II, or homolog thereof, with SAG.

23. The method of claim 22, wherein the homolog of AgI/II is selected from the group consisting of SspB, Pas, GBPC and rcnM, or a combination of any thereof.

24. The method of claim 22, wherein the inhibitor comprises a glycan.

25. The method of any one of claim 24, wherein the glycan is Galβ1-3-GalNac.

26. The method of claim 22, wherein the inhibitor comprises a peptide.

27. The method of claim 26, wherein the peptide is ETNDANVVARQL (SEQ ID NO: 10).

28. The method of claim 22, wherein the composition or formulation is administered in a form selected from the group consisting of a tooth paste, oral rinse, gel, an additive to a digestible product and a strip comprising the formulation to be applied to the teeth of a subject.

29. The method of claim 22, wherein the composition or formulation is in the form of a rinse or a solution.

30. The method of claim 22, wherein the subject is a human subject.

31. A method of identifying a compound for preventing, inhibiting or treating dental caries in a subject, wherein the compound is identified through its ability to inhibit the interaction of AgI/II, or a homolog thereof, with SAG.

32. The method of claim 29, wherein the compound binds to AgI/II, or homolog thereof.

33. The method of claim 29, wherein the compound is a peptide.


SEQUENCE LISTING (10 sequences)

SEQ ID NO: 1 - protein, 16 aa, Artificial
  SRCRP2 peptide sequence
QGRVEVLYRGSWGTVC

SEQ ID NO: 2 - protein, 9 aa, Artificial
  Consensus AgI/II aggregation sequence
VEVLXXXXW

SEQ ID NO: 3 - DNA, 39 bp, Artificial
  PCR primer
tataaccatggatgaagttacagagacaactagtacaag

SEQ ID NO: 4 - DNA, 37 bp, Artificial
  PCR primer
ttatagcggccgcaggatcctttggttttggcgttgg

SEQ ID NO: 5 - DNA, 32 bp, Artificial
  PCR primer
gcgccatggataccaatgaagcagactaccaa

SEQ ID NO: 6 - DNA, 33 bp, Artificial
  PCR primer
ataatttgcggccgctggttttgatggctccgg

SEQ ID NO: 7 - DNA, 36 bp, Artificial
  PCR primer
tataaggatccatttccactatagcagtttattagc

SEQ ID NO: 8 - DNA, 37 bp, Artificial
  PCR primer
ttatactcgagagatgcataagcaaccttattaacag

SEQ ID NO: 9 - protein, 17 aa, Artificial
  SRCRP2 peptide sequence
QGRVEVLYRGSWGTVCK

SEQ ID NO: 10 - protein, 12 aa, Artificial
  SRCR AgI/II binding peptide
ETNDANVVARQL